(19) United States
(12) Patent Application Publication
(10) Pub. No.: US 2022/0259249 A1
(43) Pub. Date: Aug. 18, 2022

(54) INHIBITORS OF ANTIBIOTIC RESISTANCE MEDIATED BY ARNT

(71) Applicants: UNIVERSITÀ DEGLI STUDI DI ROMA "LA SAPIENZA", Rome (IT); FONDAZIONE ISTITUTO ITALIANO DI TECNOLOGIA, Genova (IT)

(72) Inventors: Francesco IMPERI, Rome (IT); Fiorentina ASCENZIONI, Rome (IT); Mattia MORI, Genova (IT); Francesca GHIRGA, Genova (IT); Deborah QUAGLIO, Rome (IT); Silvia CORRADI, Rome (IT); Alessandra LO SCIUTO, Rome (IT); Bruno BOTTA, Rome (IT); Andrea CALCATERRA, Rome (IT); Roberta STEFANELLI, Catanzaro (IT)

(73) Assignees: UNIVERSITÀ DEGLI STUDI DI ROMA "LA SAPIENZA", Rome (IT); FONDAZIONE ISTITUTO ITALIANO DI TECNOLOGIA, Genova (IT)

(21) Appl. No.: 17/629,654

(22) PCT Filed: Jul. 24, 2020
(86) PCT No.: PCT/IB2020/057019

§ 371 Date: Jan. 24, 2022

(30) Foreign Application Priority Data
Jul. 25, 2019 (IT) 102019000012888

(51) Int. Cl. C07H 15/256; C07C 31/137; C07C 69/36; C07C 69/38; C07C 69/40; C07C 69/653; C07C 69/657

(52) U.S. Cl. CPC C07H 15/256; C07C 31/137; C07C 69/36; C07C 69/657; C07C 69/40; C07C 69/653; C07C 69/38

(57) ABSTRACT

The present invention relates to diterpene compounds of general formula (I) capable of contrasting the antibiotic-resistance mediated by the ArnT enzyme, to their use as a medicament, in particular for use as an adjuvant of an antibiotic therapy in the treatment of antibiotic-resistant bacterial infections. The invention relates also to associations of one or more of the compounds of formula (I) with at least another active ingredient, in particular an antibacterial agent and/or an antibiotic, and compositions comprising one or more compounds of formula (I) or the association according to the present invention and at least one pharmaceutically acceptable excipient and/or carrier as well as to products, in particular medical devices, comprising at least a compound, an association or a composition according to the present invention. Moreover, the invention relates to the use of compounds of formula (I) to sensitize a bacterium to an antibacterial agent or an antibiotic, for example, colistin (polymyxin E) or polymyxin B and to an in vivo or in vitro method for sensitizing a bacterium to an antibacterial agent or an antibiotic comprising the exposure of said bacterium to one or more compounds of formula (I) together with colistin.

### BACKGROUND ART

Antibiotic resistance represents one of the greatest threats to human health as it undermines the effectiveness of the compounds available for the treatment of the most common bacterial infections. In particular, the increase in antibiotic resistance in Gram-negative bacteria such as Pseudomonas aeruginosa, Acinetobacter baumannii and Enterobacteriaceae, is considered by the World Health Organization (WHO) to be a priority problem for which new antibiotics are urgently needed (https://www.who.int/en/news-room/detail/27-02-2017-who-publishes-list-of-bacteria-for-which-new-antibiotics-are-urgently-needed). Furthermore, the diffusion of antibiotic resistances is accompanied by a drastic reduction in the discovery of new antibiotics (Payne et al., 2007).

The polymyxins, such as colistin (polymyxin E) and polymyxin B, are increasingly used as last-line therapeutic options for the treatment of infections caused by multiresistant Gram-negative bacteria (Giamarellou, 2016; Poirel et al., 2017). The polymyxins consist of a cyclic heptapeptide ring and by a lateral tripeptide chain acylated to the N-terminal with a fatty acid tail. Polymyxins target the outer membrane of the Gram-negative bacteria with which they electrostatically interact. Electrostatic interaction occurs between amino groups of colistin and negatively charged groups present in lipid A of lipopolysaccharide (LPS), responsible for anchoring LPS to the external membrane of Gram-negative bacteria. Although the mechanism of action of colistin is not yet fully known, the colistin-lipid A interaction is believed to destabilize the outer membrane resulting in increased membrane permeability and bacterium death (Poirel et al., 2017).

However, with the use of colistin, colistin-resistant bacteria have emerged (Poirel et al., 2017). At present, colistin-resistance in Klebsiella pneumoniae, P. aeruginosa and A. baumannii appears to be limited. However, much higher resistance rates of up to 50% have been reported in the literature (Giamarellou, 2016). Furthermore, epidemiological studies of colistin resistance can provide an underestimation of the real extent of the phenomenon as to date there are no automated methods for detecting colistin resistance in the clinical setting (Jayol et al., 2018).

Colistin-resistance develops through the activation of lipid A modification systems which, once modified, is unable to interact with the antibiotic. The mechanisms responsible for these modifications include the addition of 4-amino-4-deoxy-L-arabinose (Ara4N) or phosphoethanolamine (PEtN) to lipid A. Both of these modifications reduce the net negative charge of the LPS and, therefore, the affinity of this for colistin, making the bacterium insensitive and therefore resistant to the antibiotic (Poirel et al., 2017).

More specifically, epidemiological studies and experimental evidences suggest that the prevailing molecular mechanism that confers colistin resistance in various gram-negative bacteria, including P. aeruginosa, is the enzymatic transfer of Ara4N to lipid A (Nowicki et al., 2015; Pedersen et al., 2017; Lo Sciuto e Imperi, 2018). This modification occurs through a complex series of reactions wherein the last passage is catalysed by the enzyme ArnT, an Ara4N transferase located on the cytoplasmic membrane (Petrou et al., 2016). These data suggest that ArnT inhibition may block colistin resistance in those bacteria that possess this resistance mechanism, just to name a few, P. aeruginosa, Klebsiella pneumoniae, Salmonella typhimurium, Escherichia coli (see, for example, Protein Expression and Purification, Volume 46, Issue 1, March 2006, Pages 33-39; “Purification and characterization of the L-Ara4N transferase protein ArnT from Salmonella typhimurium”, Lynn E. Bretscher et al. https://doi.org/10.1016/j.pep.2005.08.028), Burkholderia cenocepacia, Yersinia pestis, Yersinia enterocolitica, Proteus mirabilis and Salmonella enterica serovar Typhimurium (see, for example, ChemBioChem 2019, 20,2936-2948; “Synthetic Phosphodiester-Linked 4-Amino-4-deoxy-l-arabinose Derivatives Demonstrate that ArnT is an Inverting Aminoarabinosyl Transferase”, Charlotte Olagnon et al., DOI: 10.1002/cbic.201900349). Recently, the crystalline structure of ArnT has been resolved and the atomic details of its binding pocket for Ara4N have been clarified, making it possible to use in silico selection systems to identify inhibitors of ArnT, a very promising target for the development of inhibitors of colistin resistance mediated by this molecular mechanism (Petrou et al., 2016).

Potential inhibitors of colistin resistance mediated by lipid A aminoarabinosylation have been described in the literature. For example, Kline T, et al. (Synthesis of and evaluation of lipid A modification by 4-substituted 4-deoxy arabinose analogues as potential inhibitors of bacterial polymyxin resistance. Bioorg Med Chem Lett. 2008) describes an aminoarabinose analogue capable of causing a slight decrease in the degree of lipid A aminorabinosylation in an in vitro assay on membranes purified from the bacterium Salmonella typhimurium. However, said analogue has not been able to inhibit polymyxins resistance at any tested concentration (up to 90 mM).

Moreover, the international patent application WO2017083859 describes some potential inhibitors of the ArnT enzyme identified by in silico screening. These potential inhibitors were tested for the ability to inhibit the growth of Escherichia coli BL21 which expressed its ArnT enzyme or that of S. typhimurium, in the presence or absence of polymyxin B (a polymyxin with an activity equivalent to that of colistin). None of the identified compounds was able to completely inhibit the growth of the “test” strains and many of them showed a high growth inhibition activity even in the absence of antibiotic (FIGS. 8, 10, 12 and 14), suggesting that these compounds may have non-specific antibacterial activity (“off-target activity”), independent of their possible ability to inhibit the ArnT enzyme.

It is also known in the literature that some diterpene or diterpenoic derivatives, in particular derivatives of beyerenic or kauranic acid, have a certain antibacterial activity mainly against Gram-positive bacteria, such as Staphylococcus aureus (see, for example, Cai C. et al., “Two new kaurane-type diterpenoids from Wedelia chinensis (Osbeck.) Merr”, Nat. Prod. Res. 2017, 31(21), 25-31), S. aureus, Enterococcus faecalis, Bacillus subtilis and Staphylococcus epidermidis (see, for example, Drewes S. E. et al., “Antimicrobial monomeric and dimeric diterpenes from the leaves of Helichrysum tenax var tenax”, Phytochem. 2006, 67(7), 716), as well as against B subtilis, S. aureus and Mycobacterium smegmatis (see, for example, “Antimicrobial diterpenes of Croton sondenarius. II ent-Beyer-15-en-18-oic Acid”, McChesney J. D. et al., Pharm. Res. 1991, 8(10), 1243). However, none of the identified compounds is presented as useful in the treatment of antibiotic-resistant bacterial infections, in particular polymyxin-resistant, nor as a possible inhibitor of antibiotic resistance or adjuvant of antibiotics.

Therefore, the need is still felt for alternative therapeutic solutions able of effectively treating antibiotic-resistant bacterial infections and, more particularly, of increasing the efficacy and clinical duration of polymyxins, such as for example polymyxin E (colistin) and B, preventing the development of resistance to such antibiotics and/or of restoring sensitivity to said antibiotics in already resistant strains.

### SUMMARY OF THE INVENTION

By in silico screening of a vast library of natural compounds with respect to the crystallographic structure of the ArnT protein in complex with the undecaprenyl phosphate ligand, the inventors have now found that ent-beyer-15-en-18-O-oxalic acid (BBN149), a natural diterpene isolated from the leaves of the Fabiana densa var. ramulosa, and the natural or synthetic derivatives thereof are valid inhibitors/antagonists of ArnT, the enzyme responsible for the aminoribosilation of lipid A of LPS, one of the main mechanisms of antibiotic resistance in bacteria.

Therefore, the invention refers to compounds of general formula (I):

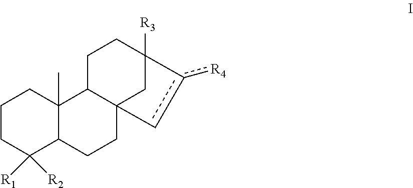

wherein
R₁ and R₂ are the same or different and independently selected among: hydrogen, C(R_{A})₃, OR_{A}, C(═O)R_{A}, C(═O)OR_{A}, CH₂OR_{A}, CH₂OC(═O)R_{A}, CH₂OC(═O)OR_{A}, CH₂OC(═O)(CH₂)ₙC(═O)OR_{A} with n=0, 1 or 2, CH₂N(R_{A})₂, C(═O)N(R_{A})₂, CH₂NHC(═O)R_{A} and CH₂C(═O)N(R_{A})₂; where R_{A} is selected among: hydrogen, alkyl, hydroxyl, monosaccharide, disaccharide and CH₂-formula (I);
R₃ is hydrogen, C(R_{B})₃ or OR_{B}; where R_{B} is selected among: hydrogen, alkyl, hydroxyl and disaccharide;
the endocyclic symbol [symbol] represents a single or double bond and, when it represents a double bond, the exocyclic symbol [symbol] binding R₄ to the carbocycle represents a single bond; R₄ is hydrogen when the exocyclic symbol [symbol] binding R₄ to the carbocycle represents a single bond; or R₄ is oxygen or methylene when the exocyclic symbol [symbol] binding R₄ to the carbocycle represents a double bond; and pharmaceutically acceptable salts thereof.
The invention also relates to their use as a medicament, in particular for use as an adjuvant of an antibiotic therapy in the treatment of bacterial infections, more particularly antibiotic-resistant bacterial infections.

Furthermore, the invention relates to associations of one or more of the compounds of formula (I) with at least one other active principle, in particular an antibacterial and/or antibiotic agent, and to compositions comprising one or more compounds of formula (I) or the association according to the present invention and at least one pharmaceutically acceptable excipient and/or carrier.

The invention also relates to products, in particular medical devices, comprising at least one compound, an association or a composition according to the present invention.

Moreover, the invention relates to the use of compounds of formula (I) to sensitize a bacterium to an antibacterial agent or an antibiotic, for example, to an antibiotic belonging to the class of polymyxins such as colistin (polymyxin E) or polymyxin B.

The invention also relates to an in vivo or in vitro method for sensitizing a bacterium to an antibacterial agent or an antibiotic which comprises the exposure of said bacterium to one or more compounds of formula (I). In particular, the in vivo or in vitro method for sensitizing a bacterium to an antibacterial agent or an antibiotic of the invention can comprise the exposure of said bacterium to one or more compounds of formula (I) together with colistin or even to the associations, compositions or products as defined above.

Finally, the invention relates to processes for the preparation of said compounds.

### DETAILED DESCRIPTION OF FIGURES

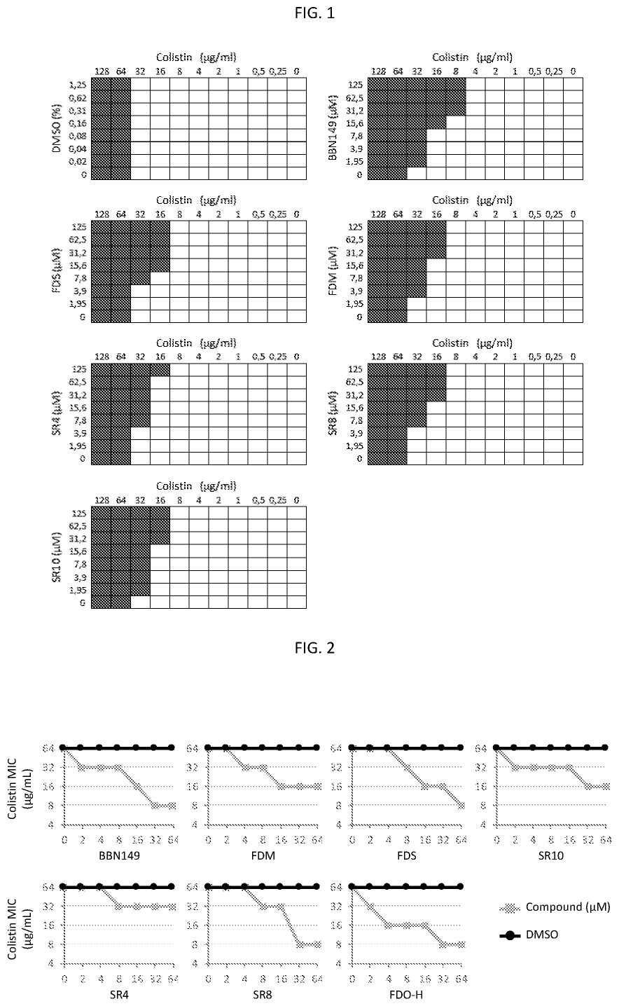

FIG. 1. Checkerboard assays to evaluate the synergistic activity between the compounds of interest, here in particular BBN149, FDS, FDM, SR4, SR8 and SR10, and colistin against the colistin-resistant strain P. aeruginosa PA14 col^{R} 5. The grey boxes highlight the combinations colistin/compound of interest capable of causing a growth inhibition of at least 90%.

FIG. 2. Graphs showing the adjuvant effect (grey line) of the compounds of interest, here in particular BBN149, FDM, FDS, SR4, SR8, SR10 and FDO-H, in increasing concentration (abscissa axis) on the MIC of colistin (ordinate axis) against the colistin-resistant strain P. aeruginosa PA14 col^{R} 5. The adjuvant effect was determined by checkerboard assay. As a control, DMSO at equivalent concentrations (0.02-0.64%; black line) was used. The data shown in the graphs are representative of at least three independent experiments.

### GLOSSARY

In the context of the present description, the term “effective amount” means amount of active compound, association or composition comprising the active compounds of the invention, sufficiently high to provide the desired benefits and at the same time low enough not to cause serious side effects.

In the context of the present description, “about” refers to the experimental error that may occur during conventional measurements. More specifically, when referring to a value it indicates ±5% of the indicated value and when referring to a range ±5% of the extremes thereof.

In the context of the present description, the terms “synergism”, “synergy”, “synergistic activity”, “activity in synergy with” and the like must be read in their broadest meaning of simultaneous action of two or more compounds which is generally expressed in a positive sense, that is, in the enhancement of the effectiveness of one or both of them. In particular, the above terms also mean “adjuvant”, “adjuvant activity” and “adjuvant activity with”, or “co-adjuvant”, respectively.

In the context of the present description, with the wording “antibiotic resistance mediated by the ArnT enzyme” is meant the phenomenon for which a bacterium is resistant to an antimicrobial drug, for example colistin, due to the action of the ArnT enzyme, the enzyme responsible for the aminoaribosylation of the lipid A component of the lipopolysaccharide (LPS). The term “mediated by” is therefore interchangeable with the terms initiated by, promoted by, caused by, exacerbated by and the like. The antibiotic resistance mediated by the ArnT enzyme can be easily identified, for example, by mass spectrometry analysis as described in greater detail in the following detailed description.

### DETAILED DESCRIPTION OF THE INVENTION

The authors of the present invention have isolated and selected diterpenes of natural origin and developed synthetic derivatives thereof capable of inhibiting the ArnT, the enzyme responsible for the aminoaribosylation of the lipid A component of the lipopolysaccharide (LPS).

In detail, a library consisting of over 1000 compounds of natural origin extracted, purified and characterized by different plants of traditional medicine and synthetic derivatives thereof was screened in silico with respect to the crystallographic structure of the ArnT enzyme in complex with the ligand undecaprenyl phosphate to identify new compounds capable of contrasting/inhibiting the activity of the ArnT enzyme and therefore enhancing the effectiveness of antibiotics in the treatment of bacterial infections caused by antibiotic-resistant Gram-negative bacteria.

By in silico screening, 18 compounds were identified (see, Table 1) which were then tested for their antibacterial activity and for their synergistic or adjuvant activity with different antibiotics, in particular colistin, against an antibiotic-resistant strain, in particular colistin-resistant, of P. aeruginosa grown in the absence or presence of a sub-inhibitory concentration of colistin.

The results obtained from the microbiological essays indicated the compound ent-beyer-15-en-18-O-oxalic acid (BNN149) and its derivatives as new potential agents capable of increasing the effectiveness of antibiotics in the treatment of antibiotic-resistant bacterial infections and, in particular, resistant to colistin.

Therefore, the present invention relates to compounds of formula (I):

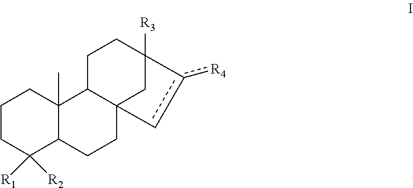

wherein
R₁ and R₂ are the same or different and independently selected among: hydrogen, C(R_{A})₃, OR_{A}, C(═O)R_{A}, C(═O)OR_{A}, CH₂OR_{A}, CH₂OC(═O)R_{A}, CH₂OC(═O)OR_{A}, CH₂OC(═O)(CH₂)ₙC(═O)OR_{A} with n=0, 1 or 2, CH₂N(R_{A})₂, C(═O)N(R_{A})₂, CH₂NHC(═O)R_{A} and CH₂C(═O)N(R_{A})₂; where R_{A} is selected among: hydrogen, alkyl, hydroxyl, monosaccharide, disaccharide and CH₂-formula (I);
R₃ is hydrogen, C(R_{B})₃ or OR_{B}; where R_{B} is selected among hydrogen, alkyl, hydroxyl and disaccharide;
the endocyclic symbol [symbol] represents a single or double bond and, when it represents a double bond, the exocyclic symbol [symbol] binding R₄ to the carbocycle represents a single bond;
R₄ is hydrogen when the exocyclic symbol [symbol] binding R₄ to the carbocycle represents a single bond; or R₄ is oxygen or methylene when the exocyclic symbol [symbol] binding R₄ to the carbocycle represents a double bond; and pharmaceutically acceptable salts thereof for the use as an adjuvant in an antibiotic therapy, in particular for the use in the treatment of bacterial infections, even more particularly antibiotic-resistant bacterial infections.

More in particular, the present invention refers to compounds of formula (I) wherein R₁ is selected among: C(═O)R_{A}, C(═O)OR_{A}, CH₂OR_{A}, CH₂OC(═O)R_{A}, CH₂OC(═O)(CH₂)ₙC(═O)OR_{A} with n=0, 1 or 2, wherein R_{A} is selected among: hydrogen, methyl, hydroxyl, monosaccharide and CH₂-formula I;

R₂ is methyl:
R₃ is selected among: hydrogen, methyl, hydroxyl and disaccharide;
the endocyclic symbol [symbol] represents a single or double bond and, when represents a double bond, the exocyclic symbol [symbol] binding R₄ to the carbocycle represents a single bond;
R₄ is hydrogen when the exocyclic symbol [symbol] binding R₄ to the carbocycle represents a single bond; or R₄ is oxygen or methylene when the exocyclic bond [symbol] binding R₄ to the carbocycle represents a double bond; and pharmaceutically acceptable salts thereof for the use as an adjuvant in an antibiotic therapy, particularly for the use in the treatment of bacterial infections, even more particularly antibiotic-resistant bacterial infections.
In one embodiment, R₁ can be selected among: CH₂OH, C(═O)OH, C(═O)OCH₃, CH₂OC(═O)(CH₂)₂C(═O)OH, CH₂OC(═O)C(═O)OH, CH₂OC(═O)CH₂C(═O)OH, C(═O)O-monosaccharide and CH₂OC(═O)(CH₂)ₙC(═O)O—CH₂-formula (I) with n=0, 1 or 2.

The terms monosaccharide and disaccharide in the context of the present invention are as commonly understood in the art and can thus indicate, respectively, any monosaccharide (for example glucose, fructose, galactose and mannose) and any disaccharide (for example sucrose, maltose, lactose, trehalose, gentiobiose and cellobiose). In one embodiment, the monosaccharide is selected between glucose and o-acetyl-glucose. In an embodiment the disaccharide is selected among sucrose, maltose and lactose.

In particular, the invention relates to a compound for the use as an adjuvant in an antibiotic therapy, wherein the compound is selected among: ent-beyer-15-en-18-ol (FDA); ent-beyer-15-en-18-O-oxalic acid (BBN149); ent-beyer-15-en-18-O-malonic acid (FDM); ent-beyer-15-en-18-O-succinic acid (FDS); glucopyranosyl ester of 4-α-13-[(2-O-β-D-glucopyranosyl-β-D-glucopyranosyl) oxy]-16β-hydroxy-entkaur-16-en-19-oic] acid (SR1); ent-16-oxo-beyeran-19-oic acid (SR2); 1,3,4,6-Tetra-O-acetyl-β-D-glucopyranosyl ester of ent-beyeran-19-oic acid (SR3); glucopyranosyl β-D ester of ent-beyeran-19-oic acid (SR4); 13-hydroxy-kaur-16-en-19-oic acid (SR5); 4-α-13-[(2-O-β-D-glucopyranosyl-β-D glucopyranosyl) kaur-16-en-19-oic) acid (SR6); methyl ester of ent-16-oxo-beyeran-19-oic acid (SR7); ent-beyeran-19-O-oxalic acid (SR8); ent-beyeran-19-ol (SR9); ent-beyeran-19-oic acid (SR10) and ent-beyeran-18-O-oxalic acid (FDO-H).

Even more in particular, the invention refers to a compound as an adjuvant in an antibiotic therapy, wherein the compound is selected among: ent-beyer-15-en-18-O-oxalic acid (BBN149); ent-beyer-15-en-18-O-malonic acid (FDM); ent-beyer-15-en-18-O-succinic acid (FDS); glucopyranosyl β-D ester of ent-beyeran-19-oic acid (SR4); ent-beyeran-19-O-oxalic acid (SR8); ent-beyeran-19-oic acid (SR10) and ent-beyeran-18-O-oxalic acid (FDO-H).

The bacterial infections mentioned above can be, for example, bacterial infections caused by Gram-negative bacteria such as Pseudomonas aeruginosa, Escherichia coli, Klebsiella pneumoniae, Klebsiella oxytoca, Enterobacter spp, Citrobacter freundii or Acinetobacter baumanni. More in particular, infections caused by Pseudomonas aeruginosa.

The antibiotic-resistant bacterial infections mentioned above can be, for example, antibiotic-resistant bacterial infections wherein the antibiotic-resistance is mediated by the enzyme transferase ArnT.

The antibiotic resistance mediated by ArnT, in particular the resistance to colistin mediated (caused, promoted, initiated, exacerbated) by ArnT, can be detected or determined according to any one of the methods known in the art, for example, using the analysis of lipid A by mass spectrometry, as for example described in (Lo Sciuto A, Martorana A M, Fernández-Piñar R, Mancone C, Polissi A, Imperi F. Pseudomonas aeruginosa LptE is crucial for LptD assembly, cell envelope integrity, antibiotic resistance and virulence. Virulence. 2018; 9(1): 1718-1733. doi:10.1080/21505594.2018.1537730).

By way of example, but in no limitative way, the analysis of mass spectrometry to determine the modification of lipid A with the aminoarabinose, catalysed by ArnT, can be carried out according to the following experimental protocol. For the extraction of lipid A, 2 mL of a bacterial culture in an early stationary phase are centrifuged at 3000×g for 10 minutes and the bacterial sediment resuspended in 400 μL of 70% isobutyric acid and 1 M ammonium hydroxide (in a 5:3 ratio). The samples are incubated for 1 hour at 100° C. and centrifuged at 2000×g for 15 minutes. The supernatants are added to 400 μL of water free of endotoxins, frozen at −80° C. and freeze-dried in a vacuum centrifuge. The resulting sediment is washed with 1 mL of methanol and lipid A is extracted from the sediment using 100 μL of a solution of chloroform, methanol and water (in a 3:1:0.25 ratio). After centrifugation at 2000×g for 15 minutes, 2 μL of supernatant are mixed with 2 μL of norharmane matrix resuspended at 10 mg/ml in a solution of chloroform and methanol (in a 2:1 ratio) and 0.5 μL of this mixture are analysed in a time-of-flight mass spectrometer with laser desorption/ionisation assisted by matrix (Matrix-assisted laser desorption/ionization Time of flight or MALDI-TOF) (5800 MALDI TOF/TOF Analyzer, Sciex). The spectral data can be analyzed with the 4000 series Explorer software version 4.1.0 (Sciex, Ontario, Canada) and used to estimate the lipid A forms based on the structures and molecular weights predicted on the basis of the literature for each bacterium. A reference strain, which does not present aminoarbinose on lipid A, may be included in the analysis as a comparison.

Therefore, the present invention also relates to compounds according to any one of the embodiment herein described for the use in the treatment of bacterial infections, more particularly of antibiotic-resistant bacterial infections, still more particularly antibiotic-resistant bacterial infections wherein the antibiotic-resistance is mediated by Arnt, wherein the antibiotic resistance mediated by Arnt is determined, for example, by extraction of the lipid A and mass spectrometry.

The above-mentioned antibiotic-resistant bacterial infections can be, for example, polymyxins-resistant bacterial infections, in particular colistin- or polymyxin B-resistant, wherein the antibiotic-resistance is mediated by the enzyme transferase ArnT.

The antibiotic resistance mediated by ArnT, in particular the resistance to colistin mediated (caused, promoted, initiated, exacerbated) by ArnT, can be identified or determined according to any of the methods known in the art, for example, using the method of microdilutions (BMD, Broth microdilution) for the determination of the minimum inhibitory concentration (MIC), test approved by the European Committee on Antibiotic Susceptibility Testing (EUCAST) and by the Clinical and Laboratory Standards Institute (CLSI) (see, European Committee on Antimicrobial Susceptibility Testing (EUCAST), Breakpoint Tables for Interpretation of MICs and Zone Diameters, Vol. 8, EUCAST, Va “xjo”, Sweden, 2018; and CLSI, Clinical and Laboratory Standards Institute, M07-A10: Methods for Dilution Antimicrobial Susceptibility Tests for Bacteria That Grow Aerobically; Approved Standard, Vol. 35, CLSI, Wayne, Pa., USA, 10^{th} edition, 2015). By way of example, MIC assays can be performed in 96-well microtitration plates. In each column of wells are aliquoted 100 μl of MH medium containing decreasing concentrations of colistin (dilutions in reason of 2 from 256 to 0.5 μg/ml) or devoid of colistin, and in each row of wells further 100 μl of MH medium containing 1×10⁶ cells/ml of each bacterial strain of interest, so as to reach a final volume of 200 μl, a concentration of bacteria equal to 0.5×10⁵ cells/ml and colistin concentrations in the range 0-128 μg/ml. The plates are incubated at 37° C. without stirring for 24 hours, and the MIC is visually assessed as the minimum concentration of colistin capable of causing absence of turbidity in the well.

Therefore, the present invention also refers to compounds according to any embodiment herein described for the use in the treatment of bacterial infections, in particular antibiotic-resistant bacterial infections, more particularly polymyxin-resistant bacterial infections, even more particularly colistin-resistant, wherein the resistance to colistin is determined by the microdilution method.

In addition to those mentioned above, infections known in the art for possessing this antibiotic-resistance mechanism, in particular to colistin, are those caused for example by Salmonella typhimurium, Burkholderia cenocepacia, Yersinia pestis and Yersinia enterocolitica, Proteus mirabilis and Salmonella enterica serovar Typhimurium.

In a preferred embodiment, the bacterial infection may be an acute or chronic pulmonary, extra-pulmonary localized or systemic infection.

The compounds of the invention can be advantageously associated in a combination product comprising one or more compounds of formula (I) (i.e. an ArnT inhibitor) and at least one other active principle.

Therefore, the present invention also refers to the association of one or more of the compounds as defined above with at least one other active principle. In one embodiment, said active principle is an antibacterial agent and/or an antibiotic.

The antibacterial agent and/or the antibiotic can be any antibacterial agent or antibiotic, in particular any antibacterial agent or antibiotic the mechanism of action of which provides for electrostatic interaction with the lipopolysaccharide of the bacterial wall. In one embodiment, the antibiotic can be a peptididic antibiotic. In a preferred embodiment, the antibiotic belongs to the class of polymyxins and is preferably colistin (polymyxin E) or polymyxin B, more preferably colistin. The efficacy of polymyxins, in particular colistin and polymyxin B, can be undermined by bacteria that have the same resistance mechanism, that is the one mediated by ArnT (see Jeannot K, Bolard A, Plésiat P. Resistance to polymyxins in Gram-negative organisms. Int J Antimicrob Agents. 2017; 49(5): 526-535. Doi: 10.1016/j.ijantimicag.2016.11.029).

Although this is not an essential feature for the purposes of therapeutic efficacy, the inventors of the present invention have also found that, when the weight ratio between compound of formula (I) and antibiotic in the association of the present invention is between 1:1-1:20, the association is able to perform its beneficial effects optimally.

The compounds and the association of the invention can be included in a pharmaceutical composition.

Therefore, the present invention also relates to compositions comprising, or consisting of, one or more compounds of the invention and at least one suitable pharmaceutically acceptable excipient or carrier, or to compositions comprising the association of the invention and at least one suitable pharmaceutically acceptable excipient or carrier. In one embodiment, the invention relates to compositions comprising one or more compounds of formula (I), at least one antibacterial agent and/or an antibiotic and at least one suitable pharmaceutically acceptable excipient or carrier.

Said excipient and/or additive may be selected among those generally known in the art such as, for example: carriers, fillers, humectants, disintegrating agents, binders, retardants, absorption accelerators, wetting agents, surfactants, adsorbents, lubricants, glidants, flavouring agents, sweeteners and/or preservatives.

The skilled in the art will be able to select appropriate additives/excipients thanks to the general knowledge in the field.

The compositions according to any of the embodiments provided in the present description, can be formulated in any form, administered by any route of administration and associated with any other component, in a variety of ways.

In particular, the compositions of the invention can be in liquid, semi-liquid, solid or semi-solid form.

The compositions are preferably, but not exclusively, administered orally or topically. Suitable liquid forms are, by way of non-limiting example, drops, emulsions, solutions, suspensions (prepared or extemporaneous), syrups and elixirs. The liquid or semi-liquid formulations may be contained in suitable delivery carriers. Suitable solid forms are, by way of non-limiting example, tablets, hard or soft capsules, pills, jellies, lozenges, powders, granulates, sachets and films. The solid dosage forms may also be coated with enteric, gastric or other coatings known in the state of the art. Suitable semi-solid forms are, by way of non-limiting example, ointments, gels, salves, creams and pastes.

In a preferred embodiment, the composition is formulated in the form of a solution, suspension, cream or ointment.

The formulations, according to any of the embodiments herein described, can be prepared according to conventional methods known to the person skilled in the art.

Moreover, the compounds, the associations or the compositions of the invention as defined above may be included in a suitable medical device useful in the treatment of bacterial infections. Non-limiting examples of medical devices suitable for the purposes of the present invention are: bandages, gauzes, patches, cotton wool, spray, prostheses, probes etc.

Therefore, the present invention also relates to medical devices comprising one or more compounds, the association or composition of the invention, wherein the compounds, the association and composition are as defined above. In particular, the invention relates to bandages, gauzes, patches, cotton wool, sprays, prostheses, probes, preferably bandages, gauzes and patches, comprising one or more compounds, the association or composition of the invention, where the compounds, the association and composition are as defined above.

The different components or active ingredients of the composition of the invention can be present in variable quantities.

Furthermore, according to any of the embodiments, the compounds, the association, the compositions and/or the medical devices of the present invention are mainly intended for use by humans, but can also be used on animals.

The present invention also relates to the use of the compounds, association or compositions as described above, for sensitizing a bacterium, in particular a gram-negative bacterium as defined above, to an antibacterial agent or an antibiotic. In particular, the antibiotic can be an antibiotic belonging to the class of polymyxins such as, for example, colistin (polymyxin E) or polymyxin B.

The present invention therefore also refers to an in vitro or in vivo method for sensitizing a bacterium to an antibacterial agent or an antibiotic which comprises the exposure of a bacterium to one or more compounds of formula (I) or to the association of the invention.

As anticipated above, in addition to the compounds of formula (I), the inventors have also isolated novel diterpenes of natural origin and designed and developed several synthetic analogues with ent-beyeranic and ent-kauranic structure of BNN149 so as to enhance the activity and selectivity thereof towards the ArnT enzyme.

Therefore, the present invention also refers to compounds of formula (I′):

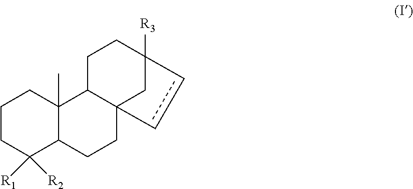

wherein
R₁ is selected among C(═O)OR_{A}, CH₂OR_{A} and CH₂OC(═O)CH₂C(═O)OR_{A}, where R_{A} is selected among hydrogen and monosaccharide (where monosaccharide is as defined above);
R₂ and R₃ are methyl; and
the symbol [symbol] represents a single or double bond; provided that when [symbol] represents a single bond R₁ is not C(═O)OH and when [symbol] represents a double bond R₁ is not —CH₂OH; and pharmaceutically acceptable salts thereof.
In an embodiment, the present invention refers to a compound of formula (I′) as defined above wherein
R₁ is selected among C(═O)OR_{A}, CH₂OR_{A} and CH₂OC(═O)CH₂C(═O)OR_{A}, where R_{A} is selected among hydrogen and monosaccharide (where monosaccharide is as defined above);
R₂ and R₃ are methyl; and
the symbol [symbol] represents a single or double bond; provided that when [symbol] represents a single bond R₁ is not C(═O)OH and when [symbol] represents a double bond R₁ is not —CH₂OH or —C(═O)OH; and pharmaceutically acceptable salts thereof.
In another embodiment, the present invention refers to a compound of formula (I′) as herein defined which does not comprise the following compounds:

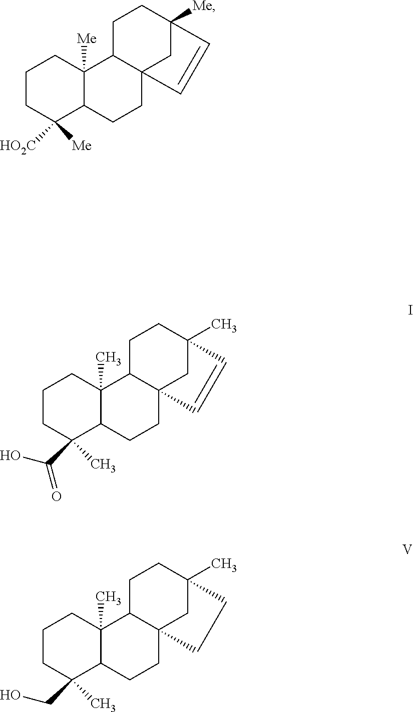 [SMILES: C[C@]12C=CC3(CCC4[C@@](C)(CCC[C@@]4(C)C(=O)O)C3CC1)C2 ; C[C@]12C=C[C@@]3(CCC4[C@@](C)(CCC[C@]4(C)C(=O)O)C3CC1)C2 ; C[C@@]12CCC3[C@@](CCC4[C@@](C)(CO)CCC[C@]43C)(CC1)C2]

In an embodiment, the present invention refers to compounds of formula (I′) wherein R₁ is selected among: CH₂OH, C(═O)OH, C(═O)OCH₃, CH₂OC(═O)(CH₂)₂C(═O)OH, CH₂OC(═O)C(═O)OH, CH₂OC(═O)CH₂C(═O)OH, C(═O)O-monosaccharide.

In particular, the present invention refers to a compound selected among: 1,3,4,6-tetra-O-acetyl-β-D-glucopyranosyl ester of ent-beyeran-19-oic acid (SR3); glucopyranosyl β-D ester of ent-beyeran-19-oic acid (SR4); and ent-beyeran-19-ol (SR9), ent-beyer-15-en-18-O-malonic acid (FDM) and ent-beyeran-18-O-oxalic acid (FDO-H).

The present invention also relates to the compounds of formula (I′) for use as a medicament. In particular, the invention also relates to the compounds of formula (I′) for use as an adjuvant in an antibiotic therapy.

The present invention also relates to the compounds described above for the use in the treatment of bacterial infections, more particularly of antibiotic-resistant bacterial infections.

Similarly to what has been described above, the invention also refers to associations, compositions and products which comprise the compounds of formula (I′), that is a subset of the products of general formula (I), their use in sensitizing antibiotic-resistant bacteria and to methods for sensitizing antibiotic-resistant bacteria to antibiotics comprising the exposure of antibiotic-resistant bacteria to said compounds, associations, compositions and/or products.

In addition, the present invention also refers to processes for the preparation of compounds of general formula (I′) as defined above. More specifically, the invention relates to processes for the preparation of the compounds indicated as SR2, SR3, SR4, SR5, SR6, SR7, SR8, SR9, SR10 and FDO-H, as taught in the following examples and illustrated in the reaction schemes (Schemes 1-11).

All the compounds of the invention have been tested in vitro on the colistin-resistant reference strain of P. aeruginosa (PA14 col^{R} 5).

The analysis of the results obtained by the microbiological essays showed that these compounds have a good ability to reduce the resistance to colistin on the PA14 col^{R} 5 strain. Moreover, the selected or synthesized compounds showed activity also towards other antibiotic-resistant strains of P. aeruginosa and for antibiotic-resistant clinical strains of other bacteria such as, for example, Klebsiella pneumoniae.

These results demonstrate that the compounds of the invention are active towards different bacterial species wherein the resistance mechanism is mediated by ArnT, just to name a few, Pseudomonas aeruginosa, Escherichia coli, Klebsiella pneumoniae, Klebsiella oxytoca, Enterobacter spp, Citrobacter freundii, Salmonella typhimurium, Burkholderia cenocepacia, Yersinia pestis and Yersinia enterocolitica, Proteus mirabilis and Salmonella enterica serovar Typhimurium. As specified above, the antibiotic resistance mediated by ArnT can be identified or determined according to any of the methods known in the art, for example, by using the method of extraction and analysis of lipid A by mass spectrometry as herein described.

Therefore, as will become clear from the following description and from the annexed examples, said compounds represent a valid alternative for the treatment of antibiotic-resistant bacterial infections, in particular when the resistance is mediated by the ArnT enzyme.

Other advantages of the compounds of the present invention will be immediately evident to the person skilled in the art on the basis of the previous description and of the examples reported below.

### EXAMPLES

The examples reported below are for illustrative purposes only and are not intended to limit the scope of the present invention. Variations and modifications of any of the embodiments described herein, which are obvious to a person skilled in the art, are included in the scope of the appended claims.

### In Silico Screening

The crystallographic structure of the ArnT protein in complex with the undecaprenyl phosphate ligand identified by the access code PDB 5F15 (Petrou V I, et al., 2016) was used as a rigid receptor in a virtual screening of a library of natural compounds consisting of about 1000 molecules, mainly extracted, isolated and characterized by plants used in traditional medicine of South America. The library is characterized by good chemical diversity and by compounds with drug-like characteristics (Lipinski et al., 1997). The visual inspection of the docking poses and the analysis of the relative scores allowed to select a restricted number of molecules that have ben tested in vitro Table 1.

TABLE 1
Structure and synergistic or adjuvant activity of the library compounds with a sub-inhibitory concentration of colistin (8 μg/ml).¹
The data were obtained on the P. aeruginosa PA14 CoI^{R}5 strain, which has a value of MIC and IC₉₀ of colistin of 64 μg/ml.

| | | Without | Synergy with colistin | |
|---|---|---|---|---|
| | | colistin | (8 μg/ml) | |
| Compound | Structure | IC₅₀ (μM) | IC₅₀ (μM) | IC₉₀ (μM) |
| BBN36 | 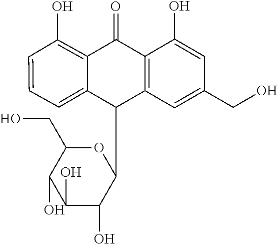 | >250 | >250 | >250 |
| BBN53 | 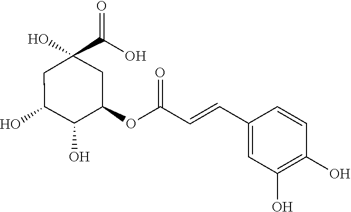 [SMILES: O=C(/C=C/c1ccc(O)c(O)c1)O[C@@H]1C[C@](O)(C(=O)O)C[C@@H](O)[C@H]1O] | >250 | >250 | >250 |
| BBN79 | 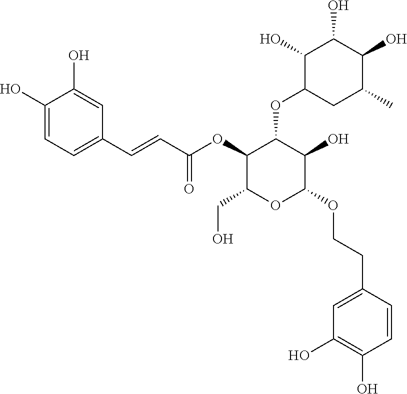 [SMILES: C[C@@H]1CC(O[C@@H]2[C@@H](O)[C@H](OCCc3ccc(O)c(O)c3)O[C@H](CO)[C@H]2OC(=O)/C=C/c2ccc(O)c(O)c2)[C@H](O)[C@H](O)[C@H]1O] | >250 | 250 | >250 |
| BBN101 | 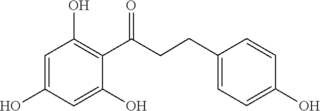 [SMILES: O=C(CCc1ccc(O)cc1)c1c(O)cc(O)cc1O] | >250 | >250 | >250 |
| BBN118 | 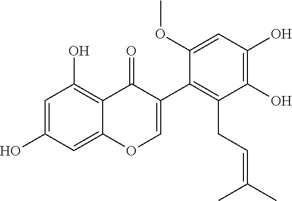 [SMILES: COc1cc(O)c(O)c(CC=C(C)C)c1-c1coc2cc(O)cc(O)c2c1=O] | >250 | 250 | >250 |
| BBN119 | 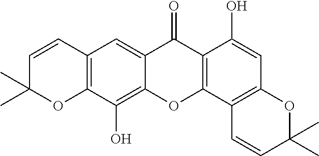 | >250 | >250 | >250 |
| BBN120 | 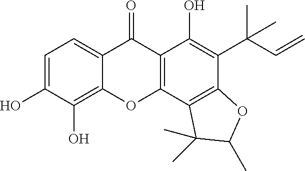 [SMILES: C=CC(C)(C)c1c2c(c3oc4c(O)c(O)ccc4c(=O)c3c1O)C(C)(C)C(C)O2] | >250 | 62.5 | >250 |
| BBN135 | 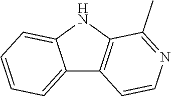 | >250 | >250 | >250 |
| BBN139 | 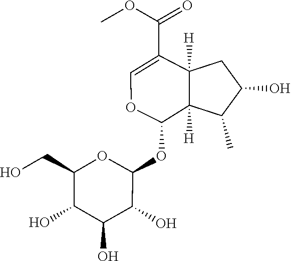 [SMILES: COC(=O)C1=CO[C@@H](O[C@@H]2O[C@H](CO)[C@@H](O)[C@H](O)[C@H]2O)[C@@H]2[C@@H](C)[C@@H](O)C[C@H]12] | >250 | >250 | >250 |
| BBN145 | 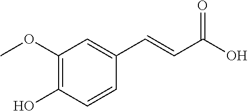 | >250 | >250 | >250 |
| BBN146 | 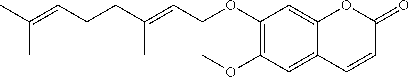 [SMILES: COc1cc2ccc(=O)oc2cc1OC/C=C(\C)CCC=C(C)C] | >250 | >250 | >250 |
| BBN147 | 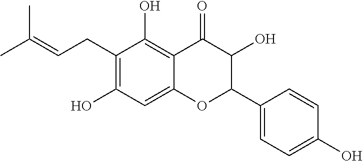 [SMILES: CC(C)=CCc1c(O)cc2c(c1O)C(=O)C(O)C(c1ccc(O)cc1)O2] | >250 | 125 | >250 |
| BBN148 | 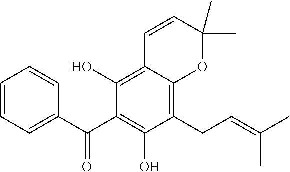 [SMILES: CC(C)=CCc1c(O)c(C(=O)c2ccccc2)c(O)c2c1OC(C)(C)C=C2] | >250 | >250 | >250 |
| BBN149 | 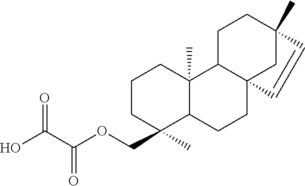 [SMILES: C[C@]12C=C[C@@]3(CCC4[C@@](C)(COC(=O)C(=O)O)CCC[C@@]4(C)C3CC1)C2] | >250 | 15.625 | 31.25 |
| BBN151 | 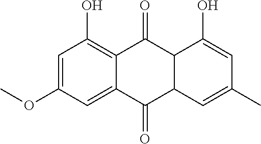 | >250 | >250 | >250 |
| BBN152 | 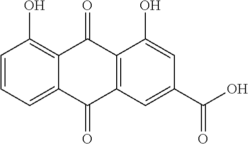 [SMILES: O=C(O)c1cc(O)c2c(c1)C(=O)c1cccc(O)c1C2=O] | >250 | >250 | >250 |
| BBN153 | 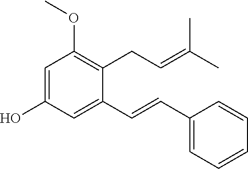 [SMILES: COc1cc(O)cc(/C=C/c2ccccc2)c1CC=C(C)C] | >250 | 15.625 | >250 |
| BBN154 | 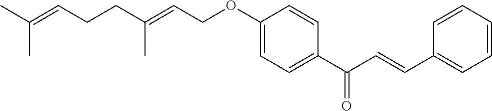 [SMILES: CC(C)=CCC/C(C)=C/COc1ccc(C(=O)/C=C/c2ccccc2)cc1] | >250 | >250 | >250 |

¹The values of IC₅₀ and IC₉₀ indicate the minimum concentration of compound able to cause at least 50% and 90% respectively of inhibition of bacterial growth.

### Evaluation of the Antibacterial Activity of the Compounds Identified by in Silico Screening

The compounds identified by in silico screening were tested for their antibacterial activity (in the absence of colistin) and for their synergistic or adjuvant activity with colistin against a colistin-resistant strain of P. aeruginosa grown in the absence or presence of a sub-inhibitory concentration of colistin (8 μg/ml). As reported in Table 1, none of the compounds showed antibacterial activity per se against P. aeruginosa. On the contrary, some compounds were found to inhibit bacterial growth in the presence of colistin (BBN79, BBN118, BBN120, BBN147, BBN149 and BBN153) with IC₅₀ values (concentration of compound necessary to inhibit bacterial growth of at least 50%) between 16 and 250 μM (Table 1), thus suggesting their synergistic or adjuvant activity with this compound. Among the various compounds with synergistic or adjuvant activity, only the BBN149 compound caused a complete inhibition of the growth of the reference strain, with an IC₅₀ value (concentration of compound necessary to inhibit bacterial growth of at least 90%) equal to about 30 μM (Table 1).

Isolation of BBN149 Analogues with Diterpene Structure Extracted from Fabiana densa var. ramulosa and Activity Essays

The BBN149 diterpene was obtained by solid-liquid extraction from the aerial parts of Fabiana densa var. ramulosa, which were collected and identified by the Department of Pharmacological and Toxicological Chemistry, University of Chile.

The aerial parts (600 g) were left to macerate in acetone for 24 h. Subsequently, the insoluble fraction was separated by filtration and new solvent was added to the solid matrix in order to increase the extraction efficiency. This operation was carried out several consecutive times. The different soluble fractions were collected together and evaporated under vacuum. The filtrate (100 g) was purified by flash chromatography. As a mobile phase, a mixture of hexane:ethyl acetate (EtOAc) was used, starting from 100% of hexane up to a ratio of 97:3 of the eluent system which allowed the isolation of the alcoholic derivative FDA (10%) and a fraction (15 g) composed of a mixture of three products. This latter fraction was subjected to a second chromatographic purification by using a gradient elution. A chloroform (CHCl₃):methanol (CH₃OH) mixture was used as the mobile phase. Using a ratio 98:2 of the eluent system the succinic derivative FDS was isolated with a yield of 20%. The increase in polarity of the mobile phase, obtained using a ratio between the two solvents equal to 97:3, allowed the elution of the malonic derivative FDM with a yield equal to 10%. The further increase in polarity through the use of an eluent mixture CHCl₃:CH₃OH:formic acid in a ratio 97:3:1% allowed the isolation of the oxalic derivative BBN149 with a yield equal to 5%.

The compounds thus obtained (BBN149, FDA, FDM and FDS) were tested for their antibacterial and synergistic or adjuvant activity with colistin as previously described. According to the results previously obtained for the compound BBN149 (Table 1), none of the compounds showed antibacterial activity per se, while three out of four compounds (BBN149, FDM and FDS) were able to inhibit bacterial growth in the presence of colistin (Table 2). Compared to the reference compound BBN149, however, the FDM and FDS compounds showed a higher IC₅₀ and an inability to cause complete inhibition of bacterial growth (values of IC₉₀>250 μM).

TABLE 2
Structure and synergistic activity with
colistin of derivatives of natural origin of the diterpene BBN149 (FDA, FDS and FDM).
Data obtained with the P. aeruginosa PA14 CoI^{R}5 strain, for which the MIC and IC₉₀ of colistin is equal to 64 μg/ml.

| | | Without | Synergy with colistin | |
|---|---|---|---|---|
| | | colistin | (8 μg/ml) | |
| Compound | Structure | IC₅₀ (μM) | IC₅₀ (μM) | IC₉₀ (μM) |
| FDA | 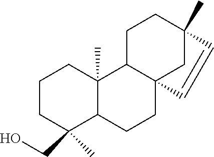 [SMILES: C[C@]12C=C[C@@]3(CCC4[C@@](C)(CO)CCC[C@@]4(C)C3CC1)C2] | >250 | >250 | >250 |
| BBN149 | 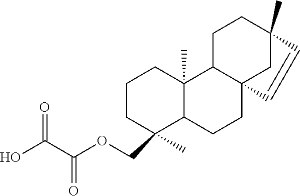 [SMILES: C[C@]12C=C[C@@]3(CCC4[C@@](C)(COC(=O)C(=O)O)CCC[C@@]4(C)C3CC1)C2] | >250 | 15.625 | 31.25 |
| FDS | 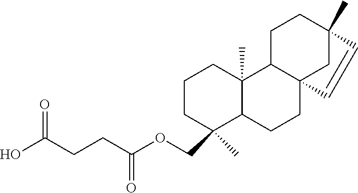 [SMILES: C[C@]12C=C[C@@]3(CCC4[C@@](C)(COC(=O)CCC(=O)O)CCC[C@@]4(C)C3CC1)C2] | >250 | 31.25 | >250 |
| FDM | 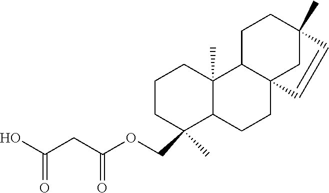 [SMILES: C[C@]12C=C[C@@]3(CCC4[C@@](C)(COC(=O)CC(=O)O)CCC[C@@]4(C)C3CC1)C2] | >250 | 62.5 | >250 |

The structural identity of the isolated compounds was determined by nuclear magnetic resonance spectroscopy (¹H-NMR and ¹³C-NMR) and mass spectrometry (MS).

### Characterization of the Components of the Extract

### Ent-beyer-15-en-18-ol (FDA)

White solid (yield 10%); m.p. 110° C.±0.5° C. [α]_{D} +29.7° (CHCl₃). ¹H NMR (CDCl₃, 400 MHz): δ (ppm)=5.69 (d, 1H, J=5.7 Hz, H-15); 5.45 (d, 1H, J=5.6 Hz, H-16); 3.41 (d, 1H, J=10.8 Hz, H-18a); 3.11 (d, 1H, J=10.8 Hz, H-18b); 0.99 (s, 3H, CH₃-17); 0.78 (s, 3H, CH₃-19); 0.77 (s, 3H, CH₃-20); ¹³C NMR (CDCl₃, 100 MHz): δ (ppm)=136.59, 135.38, 72.48, 61.34, 52.94, 49.19, 49.14, 43.81, 38.90, 37.74, 37.28, 37.16, 35.54, 33.34, 25.11, 20.38, 20.02, 18.12, 17.96, 15.76.

ESI-MS (positive) m/z: [M+Na]⁺ calculated for C₂₀H₃₂ONa 311.25, found 311.3.

### Ent-beyer-15-en-18-O-succinate (FDS)

Brown solid (yield 20%); m.p. 107.5° C.±0.5° C. [α]_{D}+14.6° (CHCl₃). ¹H NMR (CDCl₃, 400 MHz): δ (ppm)=5.67 (d, 1H, J=5.6 Hz, H-15); 5.45 (d, 1H, J=5.6 Hz, H-16); 3.88 (d, 1H, J=10.8 Hz, H-18a); 3.68 (d, 1H, J=10.08 Hz, H-18b); 2.67 (m, 4H, HOOC—CH₂CH₂—COOR); 0.99 (s, 3H, CH₃-17); 0.83 (s, 3H, CH₃-19); 0.77 (s, 3H, CH₃-20). ¹³C NMR (CDCl₃, 100 MHz): δ (ppm)=177.44, 172.28, 136.60, 135.35, 73.61, 61.24, 52.93, 50.01, 49.05, 43.75, 38.74, 37.29, 37.05, 36.70, 36.01, 33.27, 29.20, 29.06, 25.06, 20.30, 20.20, 17.91, 17.80, 15.63.

ESI-MS (negative) m/z: [M−H]⁻ calculated for C₂₄H₃₅O₄ 388.54, found 387.5; [M+Cl]⁻ calculated for C₂₄H₃₆O₄Cl 423.40, found 423.20.

### Ent-beyer-15-en-18-O-malonate (FDM)

Green oil (yield 10%); oily. [α]_{D}+25.8° (CHCl₃). ¹H NMR (CDCl₃, 400 MHz): δ (ppm)=5.67 (d, 1H, J=5.6 Hz H-15); 5.45 (d, 1H, J=5.6 Hz, H-16); 3.98 (d, 1H, J=10.8 Hz, H-18b); 3.75 (d, 1H, J=10.08 Hz, H-18a); 3.44 (s, 2H, HOOC—CH₂—COOR); 0.99 (s, 3H, CH₃-17); 0.85 (s, 3H, CH₃-19); 0.77 (s, 3H, CH₃-20). ¹³C NMR (CDCl₃, 100 MHz): δ (ppm)=169.29, 167.81, 136.66, 135.19, 74.66, 61.23, 52.92, 49.88, 49.03, 43.76, 40.53, 38.69, 37.31, 36.97, 36.77, 35.95, 33.26, 25.04, 20.30, 20.24, 17.85, 17.72, 15.62.

ESI-MS (negative) m/z: [M−H]⁻ calculated for C₂₃H₃₃O₄ 373.51; found 373.1.

### Ent-beyer-15-en-18-O-ossalate (BBN149)

White solid (yield 5%) m.p. 169.5° C.±0.5° C. [α]_{D}+10° (CHCl₃). ¹H NMR (CDCl₃, 400 MHz): δ (ppm)=5.67 (d, 1H, J=5.7 Hz, H-15,); 5.46 (d, 1H, J=5.7 Hz, H-16,); 4.10 (d, 1H, J=10.8 Hz, H-18a); 3.90 (d, 1H, J=10.8 Hz, H-18b); 0.99 (s, 3H, CH₃-17); 0.91 (s, 3H, CH₃-19); 0.79 (s, 3H, CH₃-20). ¹³C NMR (CDCl₃, 100 MHz): δ (ppm)=158.65, 157.77, 136.74, 135.13, 76.54, 61.17, 52.83, 50.08, 49.01, 43.77, 38.57, 37.36, 36.98, 36.94, 35.92, 33.21, 25.03, 20.35, 20.29, 17.78, 17.62, 15.63.

ESI-MS (negative) m/z: [M−H]⁻ calculated for C₂₂H₃₁O₄ 360.49; found 359.4.

### Characterization of the Compounds of Natural Origin

The chemical identity of the compounds examined was verified by Nuclear Magnetic Resonance (NMR). The results obtained were found to be in agreement with those reported in the literature.

Compound BBN36 (aloin or (10S)-1,8-dihydroxy-3-(hydroxymethyl)-10-[(2S, 3R, 4R, 5S, 6R)-3,4,5-trihydroxy-6-(hydroxymethyl)oxan-2-yl]-10 H-anthracen-9-one) the NMR analysis is in accordance with what reported in the literature (Jang Hoon K. et al, Journal of Enzyme Inhibition and Medicinal Chemistry, 2017, vol. 32, 78-83).

BBN53 compound (chlorogenic acid or (1S, 3R, 4R, 5R)-3-[(E)-3-(3,4-dihydroxyphenyl)prop-2-enoyl]oxy-1,4,5-trihydroxycyclohexane-1-carboxylic acid) NMR analysis is in agreement with what reported in the literature (W. Khoo et al., 2015).

BBN79 compound (verbascoside or [(2R, 3R, 4R, 5R, 6R)-6-[2-(3,4-dihydroxyphenyl)ethoxy]-5-hydroxy-2-(hydroxymethyl)-4-[(2S, 3R, 4R, 5R, 6S)-3,4,5-trihydroxy-6-methyloxan-2-yl] oxyoxane-3-yl] (E)-3-(3,4-dihydroxyphenyl)prop-2-enoate) the NMR analysis is in agreement with what reported in the literature (Venditti A. et al, 2013).

Compound BBN101 (floretin or 3-(4-hydroxyphenyl)-1-(2,4,6-trihydroxyphenyl)propan-1-one) the NMR analysis is in agreement with what reported in the literature (Qingwen Hu et al., 2018).

BBN118 compound (piscidone or 3-[3,4-dihydroxy-6-methoxy-2-(3-methylbut-2-enyl)phenyl]-5,7-dihydroxycromen-4-one) the NMR analysis is in agreement with what reported in literature (Tahara S. et al., 1991).

Compound BBN119 (Rheediaxantone A or 11,22-dihydroxy-7,7,19,19-tetramethyl-2,8,20-trioxapentacyclo [12.8.0.03,120,04,90,016,21]docosa-1(14), 3(12), 4(9), 5, 10, 15, 17, 21-octaen-13-one) the NMR analysis is in agreement with what reported in the literature (Delle Monache F. et al., 1981).

Compound BBN120 (rheediaxanthone B or NMR analysis is in accordance with what reported in the literature (Delle Monache F. et aL, 1981).

Compound BBN135 (harmane or 1-methyl-9H-pyrido[3,4-b]indole) the NMR analysis is in agreement with what reported in the literature (Z. Zhao et al., 2019).

BBN139 compound (loganine or methyl (1S, 4aS, 6S, 7R, 7aS)-6-hydroxy-7-methyl-1-[(2 S, 3 R, 4 S, 5S, 6R)-3,4,5-trihydroxy-6-(hydroxymethyl)oxan-2-yl]oxy-1, 4a, 5,6,7,7a-haxidrocrociclopenta [c] piran-4-carboxylate) the NMR analysis is in agreement with what reported in the literature (Garaev et al., 2014).

Compound BBN145 (3-hydroxy-4-methoxy cinnamic acid) the NMR analysis is in agreement with what reported in the literature (Set Byeol K. et al., 2017).

Compound BBN146 (6-methoxy-7-O-geranyl-coumarin) the NMR analysis is in agreement with what reported in the literature (Fiorito S. et al., 2018).

Compound BBN147 (6-prenyl-aromadendrine) the NMR analysis is in agreement with what reported in the literature (Toshio F. et al., 1993).

Compound BBN148 (vismiafenone b or [5,7-dihydroxy-2,2-dimethyl-8-(3-methylbut-2-enyl) cromen-6-yl]-phenylmethanone) the NMR analysis is in agreement with what reported in the literature (Delle Monache et al., 1980).

Compound BBN149 (ent-beyer-15-en-18-O-oxalic acid) the NMR analysis is in agreement with what reported in the literature (Erazo S. et al., 2002).

Compound BBN151 (physcione or 1,8-dihydroxy-3-methoxy-6-methyl-anthracene-9,10-dione) the NMR analysis is in agreement with what reported in the literature (Delle Monache F. et al., 1979) Compound BBN152 (rhein or 4,5-dihydroxy-9,10-dioxoanthracene-2-carboxylic acid) the NMR analysis is in agreement with what reported in the literature (Manshuo L. et al., 2017).

Compound BBN153 (longistiline c or 3-methoxy-4-(3-methylbut-2-enyl)-5-[(E)-2-phenylethylene]phenol) the NMR analysis is in agreement with what reported in the literature (Xing-Yue J. Et al., 2016).

Compound BBN154 (calcone19 or 4′-O-geranil-calcone) the NMR analysis is in agreement with what reported in the literature (Guglielmi P. et al., 2019).

### Design and Synthesis of BB149 Derivatives and Activity Assays

The results obtained from the microbiological tests suggested the promising role of the diterpene ent-beyerenic scaffold in modulating colistin resistance in colistin-resistant bacterial infections. Given that the BBN149 molecule emerged from the first screening as a promising hit, a first generation of diterpene derivatives was designed and synthesized with the aim of increasing the co-adjuvant ability the action of colistin and delineating the SAR (structure-activity relationship).

The ent-beyerenic scaffold differs from the ent-kaurenic one in the presence of an exocyclic double bond:

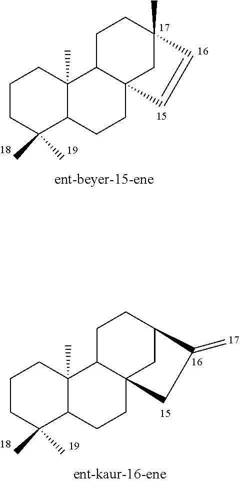

Given the possibility of obtaining diterpenes with an ent-beyeranic structure, starting from diterpenes with an ent-kaurenic structure characteristic of the diterpenes of the Stevia rebaudiana var. ramulosa, analogues of the compound BBN149 have been designed wherein the double bond is not present at positions 15 and 16 and the chiral carbon in position 4 has a different stereochemistry (R instead of 3).

Diterpene derivatives have been tested for their antibacterial and synergistic activity with colistin as previously described. No compound showed antibacterial activity per se, while three compounds (SR4, SR8 and SR10) were able to inhibit bacterial growth in the presence of colistin, with IC₅₀ values around 15-30 μM (Table 3). No compound was able to completely inhibit bacterial growth (IC₅₀ values >250 μM).

TABLE 3
Structure and synergistic or adjuvant activity with
colistin of synthetic analogues of diterpene BBN149 (SR1-SR10).
Data obtained with the P. aeruginosa PA14 CoI^{R}5 strain, for which the MIC and IC₉₀ of colistin is equal to μg/ml.

| | | Without | Synergy with colistin | |
|---|---|---|---|---|
| | | colistin | (8 μg/ml) | |
| Compound | Structure | IC₅₀ (μM) | IC₅₀ (μM) | IC₉₀ (μM) |
| SR1 | 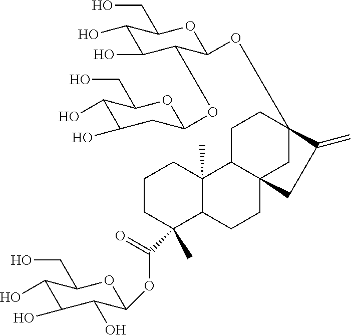 [SMILES: C=C1C[C@@]23CCC4[C@@](C)(CCC[C@@]4(C)C(=O)OC4OC(CO)C(O)C(O)C4O)C2CC[C@]1(OC1OC(CO)C(O)C(O)C1OC1CC(O)C(O)C(CO)O1)C3] | >250 | >250 | >250 |
| SR2 | 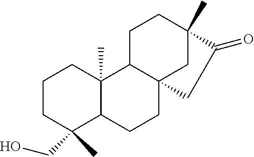 [SMILES: C[C@@]12CCC3[C@@](CCC4[C@](C)(CO)CCC[C@]43C)(CC1=O)C2] | >250 | >250 | >250 |
| SR3 | 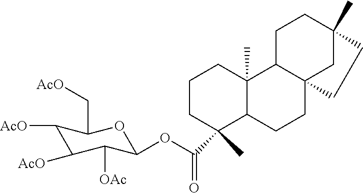 [SMILES: CC(=O)OCC1OC(OC(=O)[C@]2(C)CCC[C@@]3(C)C4CC[C@]5(C)CC[C@@]4(CCC32)C5)C(OC(C)=O)C(OC(C)=O)C1OC(C)=O] | >250 | >250 | >250 |
| SR4 | 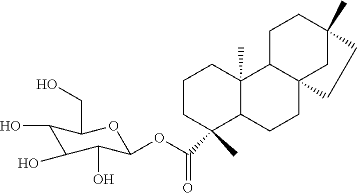 [SMILES: C[C@@]12CCC3[C@@](CCC4[C@@]3(C)CCC[C@@]4(C)C(=O)OC3OC(CO)C(O)C(O)C3O)(CC1)C2] | >250 | 15.625 | >250 |
| SR5 | 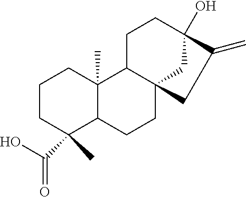 [SMILES: C=C1C[C@@]23CCC4[C@@](C)(CCC[C@@]4(C)C(=O)O)C2CC[C@]1(O)C3] | >250 | >250 | >250 |
| SR6 | 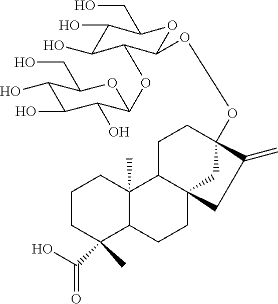 [SMILES: C=C1C[C@@]23CCC4[C@@](C)(CCC[C@@]4(C)C(=O)O)C2CC[C@]1(OOC1OC(CO)C(O)C(O)C1OC1OC(CO)C(O)C(O)C1O)C3] | >250 | >250 | >250 |
| SR7 | 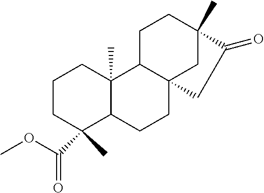 [SMILES: COC(=O)[C@]1(C)CCC[C@@]2(C)C3CC[C@@]4(C)C[C@]3(CCC21)CC4=O] | >250 | >250 | >250 |
| SR8 | 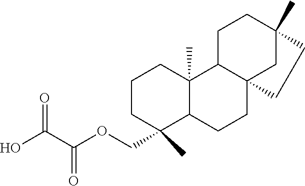 [SMILES: C[C@@]12CCC3[C@@](CCC4[C@](C)(COC(=O)C(=O)O)CCC[C@]43C)(CC1)C2] | >250 | 31.25 | >250 |
| SR9 | 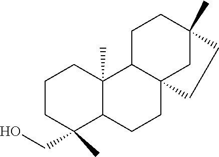 [SMILES: C[C@@]12CCC3[C@@](CCC4[C@](C)(CO)CCC[C@]43C)(CC1)C2] | >250 | >250 | >250 |
| SR10 | 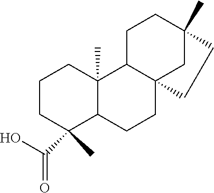 [SMILES: C[C@@]12CCC3[C@@](CCC4[C@@]3(C)CCC[C@@]4(C)C(=O)O)(CC1)C2] | >250 | 31.25 | >250 |
| FDO-H | 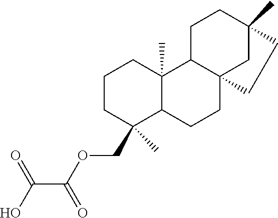 [SMILES: C[C@@]12CCC3[C@@](CCC4[C@@](C)(COC(=O)C(=O)O)CCC[C@]43C)(CC1)C2] | <250 | 7.812 | 15,625 |

### Chemical Products, Reagents and Methods of Analysis

All reagents and solvents are commercially available and have been used without further purification.

Silica gel (230-400 mesh) was used for purification by flash column chromatography. All reactions were monitored by thin layer chromatography (TLC) and F254 fluorescence silica gel plates (Sigma-Aldrich 99569) were used. The melting points were determined with a Buchi Melting Point B—454 apparatus. The ¹H and ¹³C NMR spectra were recorded with a Bruker 400 Ultra Shield™ instrument (400 MHz for ¹H NMR and 100 MHz for ¹³C NMR), using tetramethyl silane (TMS) as standard. Chemical shifts are reported in parts per million (ppm). The multiplicities were reported as follows: singlet (s), doublet (d), triplet (t) and multiplet (m). Mass spectrometry was performed with the Thermo Finnigan LXQ linear ion trap mass spectrometer, equipped with electrospray ionization (ESI). High resolution mass spectra (HR-MS) were recorded with a Bruker BioApex Fourier transform ion cyclotron resonance (FT-ICR).

### Synthetic Procedures

### Synthesis of the Compound SR2

The compound SR1 (SIG MA-ALDRICH 260-975-5) (8.68 mmol, 7.0 g) is treated with hydrobromic acid (48% HBr in water) (21 ml) and the solution, which takes on a brown colour, is left under stirring at room temperature for 16 hours. Subsequently, the precipitate is filtered and solubilized in AcOEt. The organic phase is extracted once with water and once with brine, dehydrated with anhydrous Na₂SO₄ and concentrated at a reduced pressure. The compound SR2 (7.72 mmol, 2.45 g) is obtained by crystallization with CH₃OH. (Lohoelter C. et al., 2013; Avent et al. 1989).

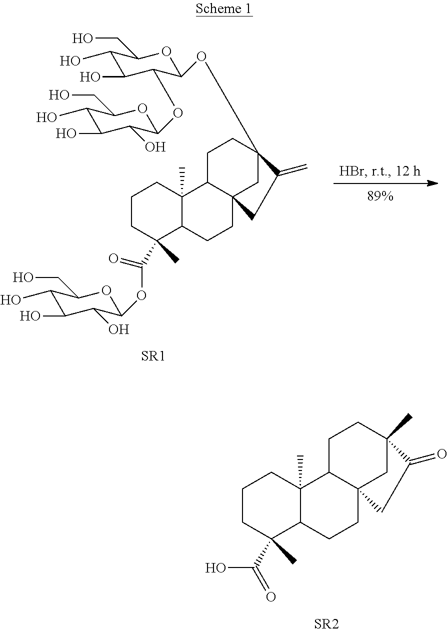 [SMILES: C=C1C[C@@]23CCC4[C@@](C)(CCC[C@@]4(C)C(=O)OC4OC(CO)C(O)C(O)C4O)C2CC[C@]1(OC1OC(CO)C(O)C(O)C1OC1OC(CO)C(O)C(O)C1O)C3 ; C[C@@]12CCC3[C@@](CCC4[C@@]3(C)CCC[C@@]4(C)C(=O)O)(CC1=O)C2]

### SR2

Brown powder (yield 89%); m.p. 230° C.±0.5° C. [α]_{D} −69.3° (EtOH). ¹H NMR (CDCl₃, 400 MHz): δ (ppm)=2.64 (dd, 1H, J=18.6 Hz e J=3.7 Hz, 1H, H-15a); 2.07 (d, 1H, J=13.3 Hz, H-3eq); 1.93-1.32 (m, 13H); 1.25 (s, 3H, CH₃-18); 1.23-1.01 (m, 4H); 0.97 (s, 3H, CH₃-17); 0.96-079 (m, 1H, H-1ax); 0.78 (s, 3H, CH₃-20); ¹³C NMR (CDCl₃, 100 MHz): δ (ppm)=222.90, 183.50, 57.17, 54.90, 54.44, 48.89, 48.61, 43.81, 41.60, 39.90, 39.63, 38.34, 37.84, 37.46, 29.11, 21.77, 20.49, 19.99, 19.01, 13.48.

ESI-HRMS (positive) m/z: [M+Na]⁻ calculated for C₂₀H₃₀O₃Na 341.20872; found 341.20902.

### Synthesis of Compound SR10

A solution containing SR2 (3.14 mmol, 1.0 g), triethylene glycol (12.5 ml), hydrazine (2.5 ml) and potassium hydroxide (KOH) (22.3 mmol, 1.25 g) is distilled at 180° C., until the removal of a volume of about 1.25 ml. After the distillation is over, the Dean-Stark is removed and the reaction is left under stirring to reflux for 22 hours at 180° C. and, subsequently, for 2 hours at 200° C. Subsequently, the reaction is brought to room temperature and 162.5 ml of distilled water are added. The solution is neutralized with glacial acetic acid (CH₃COOH) 1N. The precipitate is filtered and solubilized in diethyl ether (Et₂O). Finally, this solution is extracted 2 times with water, dehydrated with anhydrous Na₂SO₄ and concentrated under reduced pressure, thus obtaining SR10 (0.911 mmol, 277 mg). (Mosetting E. et al., 1955; Yang et al., 2012).

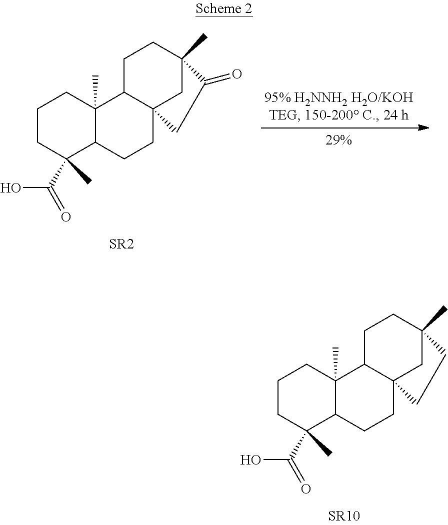 [SMILES: C[C@@]12CCC3[C@@](CCC4[C@@]3(C)CCC[C@@]4(C)C(=O)O)(CC1=O)C2 ; C[C@@]12CCC3[C@@](CCC4[C@@]3(C)CCC[C@@]4(C)C(=O)O)(CC1)C2]

### SR10

White powder (yield 29%); melting point: 190° C.±0.5° C. [α]_{D} −42.0° (CHCl₃). ¹H NMR (CDCl₃, 400 MHz): δ (ppm)=2.13 (d, 1H, J=12 Hz, H-3eq); 2.02-1.24 (m, 15H); 1.22 (s, 3H, CH₃-18); 1.18-0.95 (m, 5H); 0.93 (s, 3H, CH₃-17); 0.89 (m, 1H, H-1ax); 0.84 (s, 3H, CH₃-20). ¹³C NMR (CDCl₃, 100 MHz): δ (ppm)=184.04, 60.54, 57.52, 56.33, 45.13, 43.83, 41.54, 40.19, 40.18, 39.45, 38.36, 37.96, 37.69, 33.63, 29.19, 27.26, 21.92, 20.95, 19.09, 14.32.

ESI-HRMS (negative) m/z: [M−H]⁻ calculated for C₂₀H₃₁O₂ 303.23295; found 303.23288.

### Synthesis of Compound SR3

To a solution containing SR10 (0.986 mmol, 300 mg) in dichloromethane (CH₂Cl₂) (6.73 ml) and water (1.79 ml), tetrabutylammonium bromide (TBAB) (0.02 mmol, 6.72 mg), potassium carbonate (K₂CO₃) (3.26 mmol, 1450 mg) and acetobromo-α-D-glucose (1.36 mmol, 560 mg) are added. The solution is left under stirring to reflux for 24 hours at a temperature of 50° C. Subsequently, the aqueous phase is extracted with CH₂Cl₂ and the organic phases, in turn, are extracted twice with water, once with brine and finally dehydrated with anhydrous Na₂SO₄. Following evaporation of the solvent under reduced pressure, the compound SR3 (0.572 mmol, 363 mg) was obtained. (Klucik J. et al., 2011; Chaturvedula et al., 2011; Yang et al., 2012).

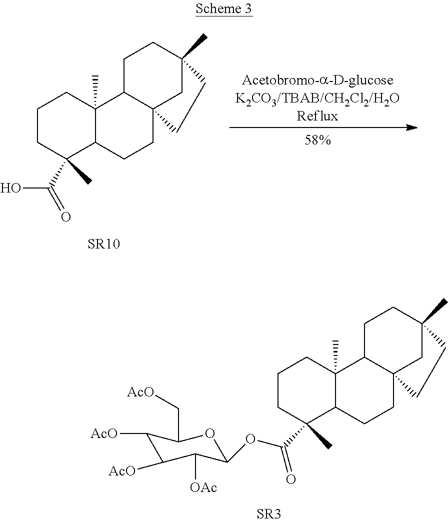 [SMILES: C[C@@]12CCC3[C@@](CCC4[C@@]3(C)CCC[C@@]4(C)C(=O)O)(CC1)C2 ; CC(=O)OCC1OC(OC(=O)[C@]2(C)CCC[C@@]3(C)C4CC[C@]5(C)CC[C@@]4(CCC32)C5)C(OC(C)=O)C(OC(C)=O)C1OC(C)=O]

### SR3

Brown powder (yield 58%); m.p. 140° C.±0.5° C. [α]_{D} −8.7° (¹H NMR (CD₃OD, 400 MHz): δ (ppm)=5.82 (d, J=8.4 Hz, 1H, H-1′); 5.34 (t, 1H, J=9.4 Hz, H-3′); 5.12-5.05 (m, 2H, H-2′e H-4′); 4.33 (dd, 1H, J=12.1 Hz, J=4.8 Hz, H-6′); 4.02 (dd, 1H, J=12.0 Hz, J=2.4 Hz, H-6″); 4.02-3.98 (m, 1H, H-5′); 2.04 (s, 3H, CH₃CO); 2.02 (s, 6H, 2×CH₃CO); 1.98 (s, 3H, CH₃CO); 1.80-1.25 (m, 16H); 1.17 (s, 3H, CH₃-17); 1.41-0.97 (m, 5H); 0.94 (s, 3H, CH₃-18); 0.77 (s, 3H, CH₃-20). ¹³C NMR (CD₃OD, 100 MHz): δ (ppm)=177.16, 172.23, 171.55, 171.23, 170.79, 92.50, 74.54, 73.57, 71.77, 69.45, 62.66, 58.53, 58.48, 57.48, 46.16, 45.18, 42.51, 41.12, 41.09, 40.34, 39.29, 39.03, 38.52, 34.82, 29.23, 27.51, 22.96, 21.89, 20.93, 20.52, 14.42.

ESI-HRMS (positive) m/z: [M+Na]⁺ calculated for C₃₄H₅₀O₁₁Na 657.32453; found 657.32481.

### Synthesis of Compound SR4

A solution of compound SR3 (0.495 mmol, 314 mg) in CH₃OH: H₂O: hexane (10:2:1) at 10% of Et₃N (7.6 ml) is stirred at room temperature for 48 hours, at the end of which the solution is concentrated under pressure and the residue obtained, the compound SR4 (0.511 mmol, 238 mg), is crystallized with Et₂O at room temperature. (Ouilmi D. et al., 1995; Chaturvedula et al., 2011; Yang et al., 2012).

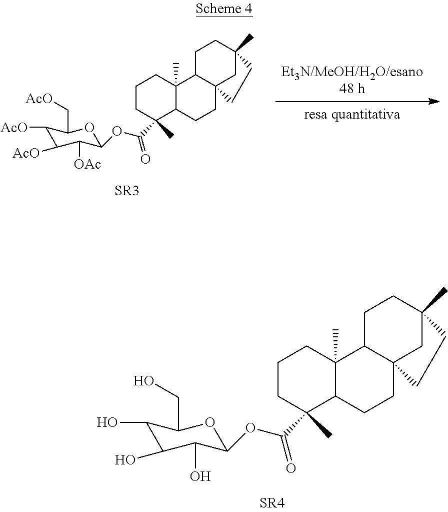 [SMILES: CC(=O)OCC1OC(OC(=O)[C@]2(C)CCC[C@@]3(C)C4CC[C@]5(C)CC[C@@]4(CCC32)C5)C(OC(C)=O)C(OC(C)=O)C1OC(C)=O ; C[C@@]12CCC3[C@@](CCC4[C@@]3(C)CCC[C@@]4(C)C(=O)OC3OC(CO)C(O)C(O)C3O)(CC1)C2]

### SR4

White powder (quantitative yield); m.p. 160° C.±0.5° C. [α]_{D} −22.7° (MeOH). ¹H NMR (CD₃OD, 400 MHz): δ (ppm)=5.42 (d, 1H, J=8.3 Hz, H-1′); 3.83 (dd, 1H, J=12.1 Hz, J=1.6 Hz, H-6′); 3.67 (dd, 1H, J=11.6 Hz, J=4.4 Hz, H-6′); 3.40-3.34 (m, 4H, H-2′, H-3′, H-4′, H-5′); 2.20-2.17 (d, 1H, J=13.2, H-3eq); 2.09-2.03 (m, 1H, H-5eq); 1.91-1.34 (m, 14H); 1.21 (s, 3H, CH₃-17); 1.18-0.97 (m, 5H); 0.94 (s, 3H, CH₃-18); 0.87 (s, 3H, CH₃-20). ¹³C NMR (CD₃OD, 100 MHz): δ (ppm)=178.27, 95.55, 78.69, 78.66, 74.03, 71.11, 62.42, 59.10, 58.56, 57.57, 46.25, 45.10, 42.75, 41.29, 41.23, 40.32, 39.37, 39.04, 38.53, 34.51, 29.24, 27.60 22.86, 21.93, 20.07, 14.42.

ESI-HRMS (positive) m/z: [M+Na]⁺ calculated for C₂₆H₄₂O₇Na 489.28227; found 489.28264.

### Synthesis of Compound SR9

To a solution of SR10 (1 mmol, 304 mg) in tetrahydrofuran (THF) (0.0854 n/l, 11.70 ml), lithium tetrahydroaluminate LiAlH₄ (9 mmol, 4.5 ml) is added dropwise. The reaction is left to reflux for about three hours at the end of which the excess of LiAlH₄ is eliminated by adding EtOAc and 20 drops of a saturated solution of Rochelle Salt. The solution is evaporated under pressure to eliminate the excess of THF, extracted with EtOAc and dehydrated with anhydrous Na₂SO₄. The SR9 compound (0.82 mmol, 238 mg) was obtained with a yield of 82%. (Batista et al., 2007; Murillo et al., 2019; Yang et al., 2012).

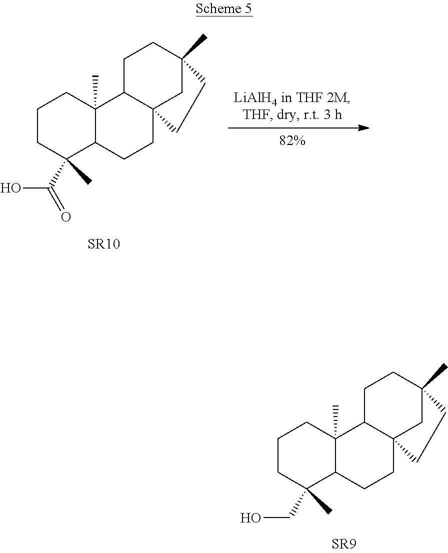 [SMILES: C[C@@]12CCC3[C@@](CCC4[C@@]3(C)CCC[C@@]4(C)C(=O)O)(CC1)C2 ; C[C@@]12CCC3[C@@](CCC4[C@](C)(CO)CCC[C@]43C)(CC1)C2]

### SR9

White powder (yield 82%); m.p. 115° C.±0.5° C.; [α]_{D} −0.7° (CHCl₃). ¹H NMR (CDCl₃, 400 MHz): δ (ppm)=3.75 (d, 1H, J=8 Hz, H-19b); 3.41 (d, 1H, J=8 Hz, H-19a,); 1.75 (d, 1H, J=12 Hz, H-3eq); 1.72-1.16 (m, 19H); 0.95 (s, 3H, CH₃-17); 0.93 (s, 3H, CH₃-19); 0.89 (s, 3H, H-20). ¹³C NMR (CDCl₃, 100 MHz): δ (ppm)=65.82, 57.78, 57.32, 57.14, 45.09, 41.78, 40.10, 39.91, 39.42, 38.67, 37.75, 37.69, 35.69, 33.74, 27.30, 27.21, 20.74, 20.50, 18.25, 15.85.

ESI-MS (positive) m/z: [M+Na]⁺ calculated for C₂₀H₃₄ONa 313.26; found 313.7.

### Synthesis of Compound SR8

To a solution containing the compound SR9 (0.207 mmol, 60 mg) in Et₂O (0.192 mmol/ml, 1.08 ml) at 0° C., oxalyl chloride (0.414 mmol, 0.207 ml) is added dropwise (ratio between starting substrate and reactive 1:2). The solution is left under stirring for 30 minutes at room temperature. The reaction is then switched off by adding H₂O until there is no more effervescence. The aqueous solution is extracted with Et₂O and the organic phase thus obtained is washed twice with water and once with brine, and dehydrated with Na₂SO₄. The residue is evaporated under pressure and purified through a flash chromatographic column using an eluent mixture CHCl₃: CH₃OH:HCOOH in a ratio 98:2:1%. The SR8 compound (0.190 mmol; 69 mg) was obtained with a yield of 92%. (Zhang X. et al., 2016).

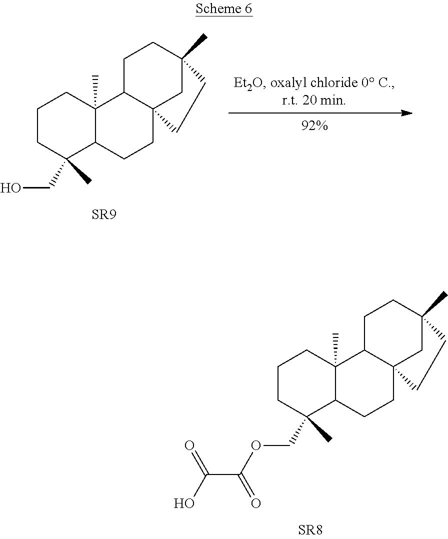 [SMILES: C[C@@]12CCC3[C@@](CCC4[C@](C)(CO)CCC[C@]43C)(CC1)C2 ; C[C@@]12CCC3[C@@](CCC4[C@](C)(COC(=O)C(=O)O)CCC[C@]43C)(CC1)C2]

### SR8

Oil (yield 92%); oily. [α]_{D} −14.3° (CHCl₃). ¹H NMR (CDCl₃, 400 MHz): δ (ppm)=4.52 (d, 1H, J=10.8 Hz, H-19a); 4.10 (d, 1H, J=10.8 Hz, H-19b); 2.04-1.3 (m, 17H); 1.16-1.03 (m, 5H); 1.00 (s, 3H, CH₃-17); 0.94 (s, 3H, CH₃-19); 0.92 (s, 3H, H-20). ¹³C NMR (CDCl₃, 100 MHz): δ (ppm)=158.59, 158.18, 71.10, 57.70, 57.15, 57.01, 45.03, 41.51, 40.03, 39.52, 39.42, 37.67, 37.66, 37.37, 36.06, 33.69, 27.42, 27.26, 20.72, 20.35, 18.05, 15.86.

ESI-MS (positive) m/z: [M+Na]⁺ calculated for C₂₂H₃₄O₄Na 385.25; found 385.3.

### Synthesis of SR6

The SR1 compound (0.62 mmol, 500 mg) is treated with a 10% solution of potassium hydroxide (KOH) (12.5 ml). The reaction is left under stirring for one hour at a temperature of 100° C. Subsequently, the reaction is cooled to room temperature, neutralized with a solution of CH₃COOH 1N and concentrated under reduced pressure. The SR6 compound (0.589 mmol, 390 mg) was obtained with a yield of 95% by crystallization with CH₃OH. (Wood J R et al., 1955, Chaturvedula et al., 2011).

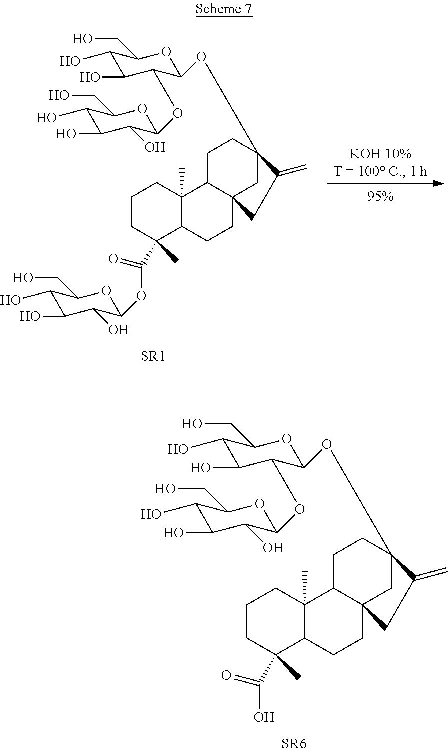 [SMILES: C=C1C[C@@]23CCC4[C@@](C)(CCC[C@@]4(C)C(=O)OC4OC(CO)C(O)C(O)C4O)C2CC[C@]1(OC1OC(CO)C(O)C(O)C1OC1OC(CO)C(O)C(O)C1O)C3 ; C=C1C[C@@]23CCC4[C@@](C)(CCC[C@@]4(C)C(=O)O)C2CC[C@]1(OC1OC(CO)C(O)C(O)C1OC1OC(CO)C(O)C(O)C1O)C3]

### SR6

White powder (yield 95%); m.p. 213° C.±0.5° C. [α]_{D} −32.5° (MeOH). ¹H NMR (DMSO-de, 400 MHz): δ (ppm)=5.08 (s, 1H, H-17); 4.74 (s, 1H, H-17b); 4.46 (d, 1H, J=8 Hz, H-1′); 4.35 (d, 1H, J=8 Hz, H-1″); 3.63-2.98 (m, 12H); 2.17-1.22 (m, 17H); 1.07 (s, 3H, H-18); 0.89 (s, 3H, H-20); 0.80-0.70 (m, 2H,); ¹³C NMR (DMSO-d₆, 100 MHz): δ (ppm)=179.50, 153.41, 104.75, 103.97, 96.36, 84.90, 82.45, 76.98, 76.63, 76.12, 76.02, 75.51, 69.87, 69.75, 60.92, 60.62, 56.56, 53.39, 47.22, 43.87, 43.09, 41.93, 41.27, 41.22, 40.66, 38.37, 36.13, 29.21, 21.95, 19.96, 19.21, 15.35.

ESI-HRMS (positive) m/z: [M+Na]⁺ calculated for C₃₂H₅₀O₁₃Na 665.31436; found 665.31488.

### Synthesis of Compound SR7

The SR2 compound (0.943 mmol, 300 mg) is treated, at 0° C., with thionyl chloride (SOCl₂) (13.6 ml) and anhydrous dimethylformamide (DMF) (0.3 ml). The solution is left under stirring for 2 hours at room temperature. Subsequently, the solvent is evaporated under reduced pressure, and anhydrous CH₃OH (54.5 ml) and triethylamine (Et₃N) (12.3 ml) are added at 0° C. The solution is left under stirring for 2 hours at room temperature and, subsequently, the residue obtained by evaporating the solvent is solubilized in CH₂Cl₂. The organic phase is extracted three times with brine and dehydrated with anhydrous Na₂SO₄, leaving it stirring overnight. The SR7 compound (0.943 moles, 313 mg) was obtained with a quantitative yield (Batista et al., 2007; Avent et al., 1989)

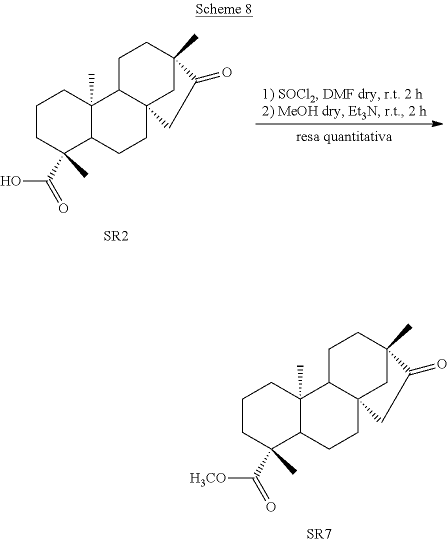 [SMILES: C[C@@]12CCC3[C@@](CCC4[C@@]3(C)CCC[C@@]4(C)C(=O)O)(CC1=O)C2 ; COC(=O)[C@]1(C)CCC[C@@]2(C)C3CC[C@@]4(C)C[C@]3(CCC21)CC4=O]

### SR7

Yellow powder (quantitative yield); m.p. 184° C.±0.5° C. [α]_{D} −46.0° (CHCl₃). ¹H NMR (CDCl₃, 400 MHz): δ (ppm)=3.63 (s, 3H, COOCH₃); 2.63 (dd, 1H, J=18.6 Hz, J=3.8 Hz, H-15ax); 2.18 (d, 1H, J=14.1 Hz, H-3eq); 2.02-1.33 (m, 15H); 1.19 (s, 3H, CH₃-18); 1.14-0.10 (m, 5H); 0.97 (s, 3H, CH₃-17); 0.89 (m, 1H, H-1ax); 0.68 (s, 3H, CH₃-20). ¹³C NMR (CDCl₃, 100 MHz): δ (ppm)=177.98, 57.22, 54.89, 54.47, 51.40, 48.86, 48.62, 43.94, 41.64, 39.97, 39.60, 38.10, 37.47, 29.00, 21.86, 20.48, 20.00, 19.10, 13.32.

ESI-HRMS (positive) m/z: [M+Na]⁺ calculated for C₂₁H₃₂O₃Na 355.22437; found 355.22466.

### Synthesis of Compound SR5

A solution containing SR1 (1.36 mmol, 1.1 g) and sodium periodate (NaIO₄) (7 mmol, 1.5 g) in water (75 ml) is left under stirring at room temperature for 16 hours. Subsequently KOH (134 mmol, 7.5 g) is added to the solution, which is left under stirring to reflux for 1 hour. Thereafter, the solution is neutralized at room temperature with CH₃COOH. The aqueous phase is extracted with Et₂O, while the organic phase is extracted with water and dehydrated with anhydrous Na₂SO₄. The compound SR5 (1.02 mmol, 324.58 mg) is obtained with a yield of 75% by crystallization with CH₃OH. (Batista et al., 2007; Avent et al., 1989).

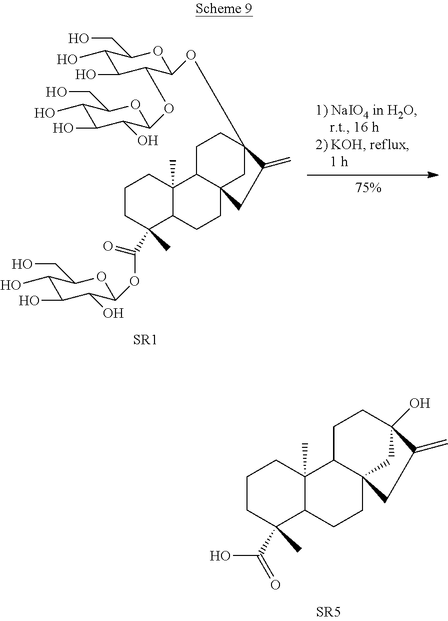 [SMILES: C=C1C[C@@]23CCC4[C@@](C)(CCC[C@@]4(C)C(=O)OC4OC(CO)C(O)C(O)C4O)C2CC[C@]1(OC1OC(CO)C(O)C(O)C1OC1OC(CO)C(O)C(O)C1O)C3 ; C=C1C[C@@]23CCC4[C@@](C)(CCC[C@@]4(C)C(=O)O)C2CC[C@]1(O)C3]

### SR5

White powder; 75% yield; melting point: 205° C. ±0.5° C.; [α]_{D} −45.7° (CDCl₃). ¹H NMR (CDCl₃, 400 MHz): δ (ppm)=4.98 (s, 1H, H-17a); 4.81 (s, 1H, H-17b); 2.22-2.07 (m, 4H); 1.95-1.30 (m, 14H); 1.23 (s, 3H, CH₃-18); 1.10-0.99 (m, 2H); 0.95 (s, 3H, CH₃-20). ¹³C NMR (CDCl₃, 100 MHz): δ (ppm)=183.30, 155.85, 103.17, 80.48, 56.99, 53.93, 47.52, 47.03, 43.71, 41.84, 41.35, 40.64, 39.62, 39.45, 37.86, 28.92, 21.92, 20.57, 19.13, 15.55.

ESI-MS (negative) m/z: [M−H]⁻ calculated for C₂₀H₃₀O₃ 317.45; found 317.50.

### Synthesis of Compound FDA-H

A solution consisting of FDA (0.329 mmol, 95 mg) and Pd/C (6.55 mg, 10%) in dry EtOH (16.4 ml) is left under stirring, under a hydrogen atmosphere (10 bar), at room temperature for 24 h. The solution is subsequently filtered, and the solvent is evaporated under pressure, obtaining the FDA-H compound (0.327 mmol, 95 mg) with a quantitative yield. (Murillo, J A et al., 2019).

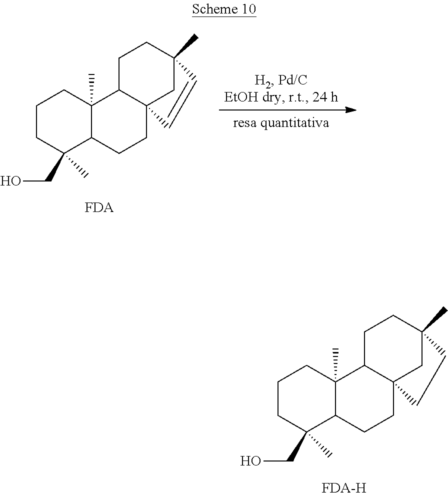

### FDA-H

White powder (quantitative yield); m.p. 108° C.±0.5° C.; [α]_{D} −4.6° (CHCl₃). ¹H NMR (CDCl₃, 400 MHz): δ (ppm)=3.40 (d, 1H, J=10.8 Hz, H-18a); 3.10 (d, 1H, J=11.2 Hz, H-18b); 2.03 (m, 1H, H-3eq.); 1.61-1.07 (m, 21H); 0.95 (s, 3H, CH₃-17); 0.93 (s, 3H, CH₃-19); 0.75 (s, 3H, CH₃-20); ¹³C NMR (CDCl₃, 100 MHz): δ (ppm)=72.45, 57.79, 56.96, 49.67, 45.06, 41.01, 40.14, 39.45, 39.41, 37.77, 37.64, 35.39, 33.90, 29.85, 27.32, 20.63, 20.08, 17.92, 17.90, 15.74.

### Synthesis of Compound FDO-H

To a solution containing the FDA-H compound (0.258 mmol, 75 mg) in Et₂O (0.192 mmol/ml, 1.34 ml) oxalyl chloride (0.516 mmol, 0.26 ml) is added dropwise at 0° C. (ratio between starting substrate and reactive 1:2). The solution is left under stirring to reflux for 30 minutes and at room temperature. The reaction is then quenched by slowly adding distilled H₂O until there is no more effervescence. The aqueous solution is extracted with Et₂O and the organic phase thus obtained is washed twice with water and once with brine, and dehydrated on Na₂SO₄. The residue is evaporated under pressure and purified through a flash chromatographic column using an eluent mixture CHCl₃: CH₃OH: HCOOH in a ratio 98:2:1%. The FDO-H compound (0.196 mmol; 71 mg) was obtained with a yield of 76%. (Zhang, X. et al., 2016).

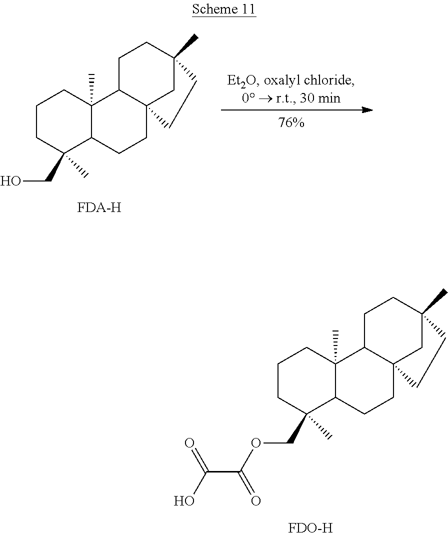

### FDO-H

Light yellow oil (yield 76%); [α]_{D} −5° (CHCl₃); ¹H NMR (CDCl₃, 400 MHz): δ (ppm)=4.10 (d, 1H, J=10.8 Hz, H-18a); 3.90 (d, 1H, J=10.8 Hz, H-18b); 2.03 (m, 1H, H-3eq.); 0.96 (s, 3H, CH₃-17); 1.70-1.07 (m, 21H); 0.93 (s, 3H, CH₃-19); 0.89 (s, 3H, CH₃-20). ¹³C NMR (CDCl₃, 100 MHz): δ (ppm)=158.5, 157.80, 57.67, 56.88, 50.62, 44.98, 40.81, 40.04, 39.44, 39.12, 37.75, 37.71, 36.95, 35.83, 33.84, 29.85, 27.27, 20.57, 20.46, 17.62, 17.59, 15.69. ESI-HRMS (negative) m/z: [M−H]⁻ calculated for C₂₂H₃₃O₄ 361.23843; found 361.23816.

Biological Activity Assays

Bacterial Growth Inhibition Assays

For the evaluation of the antibacterial activity and synergy with colistin of the compounds under examination, a colistin resistant P. aeruginosa strain (PA14 col^{R} 55) with a minimum inhibitory concentration (MIC) of colistin equal to 64 μg/ml, previously obtained by in vitro growth in the presence of increasing concentrations of colistin (Lo Sciuto e Imperi, 2018). This strain over-expresses the am genes responsible for the modification of lipopolysaccharide by the addition of L-amino-arabinose.

The strain was grown in Mueller-Hinton (MH) medium for 8 hours, and inoculated at a cell density of ˜10⁶/ml in MH medium supplemented or not with colistin at a concentration of 16 μg/ml. One hundred μl of the bacterial cultures were aliquoted in the wells of a 96-well microtitration plate, containing 100 μl of MH medium supplemented with decreasing concentrations of the compound to be tested (500, 250, 125, 62.5, 31.25, 16.625, 8.313 or 4.156 μM) or without compound, so as to bring the bacterial concentration to 5×10⁵ cells/ml and that of the compounds to 8 μg/ml for colistin and in the range 0-250 μM for the compounds under examination, in a final volume of 200 μl. The microtitration plates were incubated at 37° C. without stirring for 24 hours. Bacterial growth was assessed by measuring absorbance at 600 nm (A₆₀₀) in a microplate reader (Victor 2^{V}, Perkin-Elmer). Since the compounds are dissolved in DMSO at a concentration of 10 mM, in each assay the bacterial growth is also analyzed in the presence of DMSO at concentrations equivalent to those present in the samples that contain the compounds (0-2.5%) and in the presence or absence of 8 μg/ml colistin. The 50% or 90% inhibitory concentration (IC₅₀ and IC₉₀) for each compound was determined as the minimum concentration of the compound capable of causing at least 50% or 90% of bacterial growth inhibition compared to control cultures grown under the same conditions in the presence of an equivalent concentration of DMSO. For each compound three independent experiments were conducted, and the averages of the values obtained in the three experiments are considered to determine the values of IC₅₀ e IC₉₀.

### Checkerboard Essays

To further confirm the synergistic activity with colistin of the diterpene compounds under examination, checkerboard essays were carried out, which allow to evaluate the antibacterial activity of the compounds under examination (diterpene derivative and colistin) using different combinations of concentration of the two compounds (FIG. 1). For these essays the most promising compounds were selected based on the previously obtained results (BBN149, FDM, FDS, SR4, SR8 and SR10; Tables 2-3). Again, the greatest synergistic activity was found in the compound BBN149, which was able to reduce the minimum inhibitory concentration (MIC) of colistin for the colistin-resistant strain of P. aeruginosa by 8 times (64 to 8 μg/ml) at concentrations of BBN149≥31 μM, and 4 times (64 to 16 μg/ml) at concentrations of BBN149≥16 μM (FIG. 1). Similarly, the FDO-H compound reduces the MIC of colistin by 8 times at concentrations ≥31 μM while decreasing it by 4 times at concentrations ≥4 μM. Compounds FDS, FDM, SR8 and SR10 showed slightly less activity, managing to cause at most a 4-fold reduction in the MIC of colistin (64 to 16 μg/ml) at minimum concentrations of compound between 16 and 31 μM. Compound SR4 was the least active, being able to reduce the MIC of colistin by 4 times only at a concentration equal to 125 μM (FIG. 1).

The checkerboard essays were performed in microtiter plates with 96 wells, aliquoting in each column of wells 50 μl of MH medium containing decreasing concentrations of colistin (dilutions in reason of 2 from 384 to 1.5 μg/ml) or without colistin, and in each row of wells further 50 μl of MH medium containing decreasing concentrations of compound of interest (dilutions in reason 2 from 375 to 11.7 μg/ml) or without compound. Finally, 50 μl of the inoculum of the PA14 col^{R} 5 strain were added to each well at a concentration of ˜1.5×10⁶ cells/ml in MH medium, so as to reach a final volume of 150 μl, a bacteria concentration equal to 0.5×10⁵ cells/ml, colistin concentrations in the range 0-128 μg/ml and concentrations of compounds of interest in the range 0-125 μM. Plates were incubated at 37° C. without shaking for 24 hours, and bacterial growth was assessed by measuring the A₆₀₀ in a microplate reader (Victor 2^{V}, Perkin-Elmer). Three independent experiments were conducted for each compound, and the averages of the values obtained in the three experiments were considered to determine the IC₉₀ values.

### Minimum Inhibitory Concentration (MIC) Essays: Activity Spectrum and Specificity of the Compound BBN149

To evaluate the spectrum of activity and the specificity of the compound BBN149, which was found to be the most effective compound in enhancing the activity of colistin (Tables 1-3 and FIG. 1), MIC essays were performed using different strains of P. aeruginosa, both resistant and sensitive to colistin, and colistin-resistant clinical strains of another Gram-negative bacterium, Klebsiella pneumoniae. All the colistin-resistant strains used depend on the aminoarabinosylation of lipid A as a mechanism of resistance to colistin (Lo Sciuto e Imperi, 2018; Esposito et al., 2018). As reported in Table 5, the compound BBN149 was able to reduce the MIC of colistin, by a value between 4 and 16 times, in all the colistin-resistant strains analyzed, both of P. aeruginosa and of K. pneumoniae. Furthermore, the compound showed no relevant activity on colistin-sensitive strains (Table 5). Overall, these data demonstrate that the BBN149 compound is able to specifically interfere with the mechanism of resistance to colistin in various Gram-negative bacteria. MIC essays were performed in 96-well microtitration plates, using previously characterized P. aeruginosa and K. pneumoniae strains (Lo Sciuto e Imperi, 2018; Esposito et al., 2018). In each column of wells were aliquoted 100 μl of MH medium containing decreasing concentrations of colistin (dilutions in reason 2 from 256 to 0.5 μg/ml) or without colistin, and in each row of wells further 100 μl of MH medium containing ˜1×10₆ cells/ml of each bacterial strain of interest and BBN149 or DMSO as a control at a concentration of 120 μM or 1.2% respectively, so as to reach a final volume of 200 μl, an equal concentration of bacteria at 0.5×10⁵ cells/ml, colistin concentrations in the range 0-128 μg/ml and concentrations of BBN140 or DMSO equal to 60 μM or 0.6% respectively. The plates were incubated at 37° C. without stirring for 24 hours, and the MIC was visually evaluated as the minimum concentration of colistin capable of causing absence of turbidity in the well. At least three independent experiments were conducted for each strain.

TABLE 5
MIC of colistin for different strains of
P. aeruginosa and K. pneumoniae in the presence
of 60 μM BBN149 or 0.6% DMSO as a control.

| | | MIC (μg/ml) | |
|---|---|---|---|
| Specie | Strain | BBN149 | DMSO |
| P. aeruginosa | PA14 col^{R} 5 | 8 | 64 |
| | PA14 | 1 | 0.5 |
| | KK1 col^{R} 1 | 8 | 128 |
| | KK1 | 1 | 0.5 |
| | KK27 col^{R} 6 | 4 | 64 |
| | KK27 | 1 | 0.5 |
| | TR1 col^{R} 6 | 4 | 16 |
| | TR1 | 1 | 0.5 |
| K. pneumoniae | KP-Mo-3 | 16 | 128 |
| | KP-Mo-5 | 8 | 128 |
| | KP-Mo-6 | 4 | 32 |
| | KP-Mo-11 | 8 | 64 |
| | KP-Mo-16 | 8 | 64 |

### Cell Viability Test

The cytotoxic potential of the compounds was evaluated on cell cultures in vitro. For these essays, the most promising compounds were selected based on their antibacterial activity in synergy with colistin. In particular, for the compounds BBN149, FDM, FDS, SR4, SR8 and SR10, cytotoxic activity was determined on human epithelial cells of bronchial origin, 16HBE (Cozens et al, 1994). All compounds were tested in the concentration range of 1.95 μM to 250 μM with an exposure of 18 hours. The results are reported in the table as viability (%) compared to untreated cells (Table 4). Cell viability of control samples treated only with the solvent (DMSO) was equal to 97.11% (±7.65) regardless of the concentration used, in the range between 2.5% and 0.02% (1/2 serial dilutions). Although almost all the compounds show a reduction in cell viability from 15% to 35% at the highest concentrations (250 μM), at concentrations active against P. aeruginosa, cell viability is reduced by a maximum of about 10%. Compounds SR4 and SR10 show a total reduction of cell viability, exclusively at the highest concentration (250 μM). This result can be explained by the reduced solubility of the compound in aqueous media and therefore by the possible precipitation in the cell culture medium.

To evaluate the cytotoxic potential of the compounds under examination, the MTT (3-(4,5-dimethylthiazol-2-yl)-2,5-diphenyltetrazolium) reduction test, commonly used to monitor cell viability was used following the exposure to potentially cytotoxic agents. The essay is based on the reduction of MTT to formazan, a compound that assumes a blue colour which can be evaluated by spectrophotometric reading at a wavelength of 570 nm. For the essay we used the 16HBE cell line. 16HBE cells are derived from human bronchi and their production is reported in Cozens et al (1994). The essay was performed as follows: 3×10₄ cells per well were seeded in 96-well multi-well plates and incubated (37° C., 5% CO₂) to allow adhesion to the bottom of the plate (8-12 hr); the compounds dissolved in DMSO at a concentration of 10 mM were added to a final concentration of 250 μM and diluted 1/2 up to 1.95 μM in the corresponding wells; similarly control samples were treated with 2.5% DMSO (equal to the amount of DMSO present in the samples treated with the compounds at 250 μM) and serially diluted 1/2 up to 0.019%; the cells were then incubated for 18 hours (37° C., 5% CO₂); MTT 0.5 mg/ml was added to each well of the plate and the plate was incubated again for 3 hours (37° C., 5% CO₂); after incubation, the supernatant was removed and the formazan was solubilized in DMSO; the quantity of formazan produced was determined by spectrophotometric reading at a wavelength of 570 nm. Each test was performed in duplicate and three independent replicas were made.

Cell viability was calculated as follows:

C_{OD570}=OD₅₇₀ of sample−OD₅₇₀ of wells without cells (white)

Reduction (%)=(C_{OD570}NT−C_{OD570}T)/C_{OD570}NT×100

Viability=100−reduction (%)

where: NT indicates the samples of cells non-threated with the compounds; T the samples of cells treated with the compounds at the different concentrations.

The mean cell viability values were calculated using the Excel mean and standard deviation functions.

TABLE 4
Average percentage of cell viability (±standard deviation)

| μM | FDM | BBN149 | FDS | SR4 | SR8 | SR10 | FDO-H | DMSO |
|---|---|---|---|---|---|---|---|---|
| 250 | 85.01 ± 2.40 | 65.51 ± 4.05 | 86.12 ± 3.18 | −1.11 ± 2.02 | 80.16 ± 2.01 | 1.47 ± 2.29 | 76.51 ± 12.64 | 72.15 ± 19.63 |
| 125 | 100.48 ± 12.78 | 71.99 ± 5.18 | 105.88 ± 1.31 | 106.23 ± 5.52 | 78.02 ± 7.46 | 92.07 ± 5.47 | 98.42 ± 16.71 | 76.74 ± 6.62 |
| 62.5 | 106.63 ± 2.87 | 76.33 ± 2.65 | 102.15 ± 3.15 | 99.27 ± 2.43 | 85.68 ± 6.99 | 99.80 ± 4.30 | 97.14 ± 12.19 | 85.58 ± 9.56 |
| 31.25 | 113.17 ± 8.95 | 86.07 ± 5.30 | 102.95 ± 6.74 | 101.69 ± 4.39 | 90.55 ± 4.17 | 99.52 ± 9.52 | 93.65 ± 11.27 | 87.92 ± 5.29 |
| 15.63 | 107.42 ± 0.01 | 90.80 ± 6.31 | 99.77 ± 6.58 | 97.27 ± 5.61 | 92.64 ± 6.57 | 97.98 ± 1.66 | 94.06 ± 3.88 | 92.29 ± 5.70 |
| 7.81 | 97.93 ± 2.17 | 86.13 ± 2.37 | 93.53 ± 2.01 | 92.44 ± 2.11 | 90.33 ± 4.26 | 101.00 ± 7.12 | 100.25 ± 23.84 | 90.21 ± 3.93 |
| 3.91 | 96.70 ± 1.60 | 90.17 ± 7.35 | 95.34 ± 8.28 | 92.31 ± 3.26 | 97.84 ± 10.19 | 104.95 ± 6.36 | 94.57 ± 12.67 | 97.03 ± 9.44 |
| 1.95 | 102.92 ± 7.31 | 94.15 ± 6.34 | 98.93 ± 5.38 | 96.35 ± 9.42 | 100.17 ± 14.21 | 105.21 ± 6.73 | 106.31 ± 17.23 | 105.83 ± 6.65 |

1. An adjuvant in antibiotic therapy comprising a compound of formula (I): 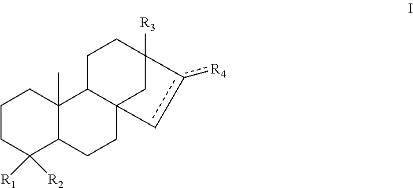
wherein
R₁ and R₂ are the same or different and independently selected among: hydrogen, C(R_{A})₃, OR_{A}, C(═O)R_{A}, C(═O)OR_{A}, CH₂OR_{A}, CH₂OC(═O)R_{A}, CH₂OC(═O)OR_{A}, CH₂OC(═O)(CH₂)ₙC(═O)OR_{A} with n=0, 1 or 2, CH₂N(R_{A})₂, C(═O)N(R_{A})₂, CH₂NHC(═O)R_{A} and CH₂C(═O)N(R_{A})₂; where R_{A} is selected among: hydrogen, alkyl, hydroxyl, monosaccharide, disaccharide and CH₂-formula (I);
R₃ is hydrogen, C(R_{B})₃ or OR_{B}; where R_{B} is selected among: hydrogen, alkyl, hydroxyl, and disaccharide;
the endocyclic symbol [symbol] represents a single or double bond and, when it represents a double bond, the exocyclic symbol [symbol] binding R₄ to the carbocycle represents a single bond;
R₄ is hydrogen when the exocyclic symbol [symbol] binding R₄ to the carbocycle represents a single bond; or
R₄ is oxygen or methylene when the exocyclic symbol [symbol] binding R₄ to the carbocycle represents a double bond;
and pharmaceutically acceptable salts.

2. The adjuvant in antibiotic therapy comprising the compound according to claim 1, wherein R₁ is selected among: C(═O)R_{A}, C(═O)OR_{A}, CH₂OR_{A}, CH₂OC(═O)R_{A}, CH₂OC(═O)(CH₂)ₙC(═O)OR_{A} with n=0, 1 or 2, where R_{A} is selected among: hydrogen, methyl, hydroxyl, monosaccharide and CH₂-formula I;
R₂ is methyl;
R₃ is selected among: hydrogen, methyl, hydroxyl and disaccharide;
the endocyclic symbol [symbol] represents a single or double bond and, when it represents a double bond, the exocyclic symbol [symbol] binding R₄ to the carbocycle represents a single bond;
R₄ is hydrogen when the exocyclic symbol [symbol] binding R₄ to the carbocycle represents a single bond; or R₄ is oxygen or methylene when the exocyclic symbol [symbol] binding R₄ to the carbocycle represents a double bond.

3. The adjuvant in antibiotic therapy comprising the according to claim 2, wherein R₁ is selected among: CH₂OH, C(═O)OH, C(═O)OCH₃, CH₂OC(═O)(CH₂)₂C(═O)OH, CH₂OC(═O)C(═O)OH, CH₂OC(═O)CH₂C(═O)OH, C(═O)O-monosaccharide and CH₂OC(═O)(CH₂)ₙC(═O)O—CH₂-formula I with n=0, 1 or 2.

4. The adjuvant in antibiotic therapy comprising the compound according to claim 1, wherein the compound is selected among: ent-beyer-15-en-18-ol (FDA); ent-beyer-15-en-18-O-oxalic acid (BBN149); ent-beyer-15-en-18-O-malonic acid (FDM); ent-beyer-15-en-18-O-succinic acid (FDS); glucopyranosyl ester of 4-α-13-[(2-O-β-D-glucopyranosyl-β-D-glucopyranosyl)oxy]-16β-hydroxy-entkaur-16-en-19-oic] acid (SR1); ent-16-bone-beyeran-19-oic acid (SR2); 1,3,4,6-tetra-O-acetyl-β-D-glucopyranosyl ester of ent-beyeran-19-oic acid (SR3); glucopyranosyl β-D ester of ent-beyeran-19-oic acid (SR4); 13-hydroxy-kaur-16-en-19-oic acid (SR5); 4-α-13-[(2-O-β-D-glucopyranosyl-β-D glucopyranosyl) kaur-16-en-19-oic) acid (SR6); methyl ester of ent-16-oxo-beyeran-19-oic acid (SR7); ent-beyeran-19-O-oxalic acid (SR8); ent-beyeran-19-ol (SR9); ent-beyeran-19-oic acid (SR10) and ent-beyeran-18-O-oxalic acid (FDO-H).

5. The adjuvant in antibiotic therapy comprising the compound according to claim 4, wherein the compound is selected among: ent-beyer-15-en-18-O-oxalic acid (BBN149); ent-beyer-15-en-18-O-malonic acid (FDM); ent-beyer-15-en-18-O-succinic acid (FDS); glucopyranosyl β-D ester of ent-beyeran-19-oic acid (SR4); ent-beyeran-19-O-oxalic acid (SR8); ent-beyeran-19-oic acid (SR10) and ent-beyeran-18-O-oxalic acid (FDO-H).

6. A method for treating bacterial infections comprising administering a therapeutically effective amount of the adjuvant in antibiotic therapy comprising the compound of claim 1 to a subject in need thereof.

7. The method of claim 6, wherein the bacterial infections are antibiotic-resistant bacterial infections.

8. A compound of formula (I′): 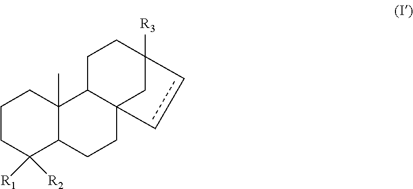
wherein the compound is selected among: 1,3,4,6-tetra-O-acetyl-β-D-glucopyranosyl ester of ent-beyeran-19-oic acid (SR3); glucopyranosyl β-D ester of ent-beyeran-19-oic acid (SR4); and ent-beyeran-19-ol (SR9), ent-beyer-15-en-18-O-malonic acid (FDM) and ent-beyeran-18-O-oxalic acid (FDO-H).

9. (canceled)

10. An adjuvant in antibiotic therapy comprising the compound of claim 8.

11. A method of treating bacterial infections comprising a therapeutically effective amount of the compound of claim 8 to a subject in need thereof.

12. The method of claim 11, wherein the bacterial infections are antibiotic-resistant bacterial infections.

13. The method according to claim 6, wherein the bacterial infections are caused by a Gram-negative bacterium selected among Pseudomonas aeruginosa, Escherichia coli, Klebsiella pneumoniae, Klebsiella oxytoca, Enterobacter spp, Citrobacter freundii, Salmonella typhimurium, Burkholderia cenocepacia, Yersinia pestis, Yersinia enterocolitica, Proteus mirabilis and Salmonella enterica serovar Typhimurium.

14. The method according to claim 7, wherein the antibiotic-resistant bacterial infections are bacterial infections wherein the antibiotic-resistance is mediated by the aminoribosiltransferase (ArnT) enzyme as measured by mass spectrometry.

15. The method according to claim 6, wherein the infection is an acute or chronic pulmonary, extrapulmonary localized or systemic infection.

16. A composition comprising the adjuvant in antibiotic therapy comprising the compound according to claim 1 and at least another active principle.

17. The composition according to claim 16 wherein the active principle is an antibacterial agent and/or an antibiotic.

18. The composition according to claim 16, wherein the weight ratio between compound of formula (I) and antibacterial agent and/or antibiotic is between 1:1-1:20.

19. The composition according to claim 17, wherein the antibiotic belongs to the class of polymyxins and is preferably colistin (polymyxin E) or polymyxin B.

20. The composition according to claim 16, further comprising a pharmaceutically acceptable excipient or carrier.

21. The composition according to claim 20 which is in liquid, solid or semisolid form.

22. A bandage, gauze, patch, cotton wool, spray, prostheses or probes comprising the adjuvant in antibiotic therapy comprising the compound according to claim 1.

23. (canceled)

24. A method for sensitizing a bacterium to an antibacterial agent or an antibiotic comprising exposing the bacterium to the adjuvant in antibiotic therapy comprising the compound of claim 1.

25. The method according to claim 11, wherein the bacterial infections are caused by a Gram-negative bacterium selected among Pseudomonas aeruginosa, Escherichia coli, Klebsiella pneumoniae, Klebsiella oxytoca, Enterobacter spp, Citrobacter freundii, Salmonella typhimurium, Burkholderia cenocepacia, Yersinia pestis, Yersinia enterocolitica, Proteus mirabilis and Salmonella enterica serovar Typhimurium.

26. The method according to claim 12, wherein the antibiotic-resistant bacterial infections are bacterial infections wherein the antibiotic-resistance is mediated by the aminoribosiltransferase (ArnT) enzyme as measured by mass spectrometry.

27. The method according to claim 11, wherein the infection is an acute or chronic pulmonary, extrapulmonary localized or systemic infection

28. A composition comprising the compound according to claim 8 and at least another active principle.

29. The composition according to claim 27, further comprising a pharmaceutically acceptable excipient or carrier.

30. The composition according to claim 28, in liquid, solid or semisolid form.

31. A bandage, gauze, patch, cotton wool, spray, prostheses or probes comprising the compound according to claim 8.

32. A method for sensitizing a bacterium to an antibacterial agent or an antibiotic comprising exposing the bacterium to the compound of claim 8.
